(19) United States
(12) Patent Application Publication
(10) Pub. No.: US 2018/0002285 A1
(43) Pub. Date: Jan. 4, 2018

(54) STERICALLY HINDERED ALKYL AND OXYALKYL AMINE LIGHT STABILIZERS

(71) Applicant: 3M INNOVATIVE PROPERTIES COMPANY, St. Paul, MN (US)

(72) Inventors: Thomas P. Klun, Lakeland, MN (US); Mark A. Roehrig, Stillwater, MN (US); Joseph C. Spagnola, Woodbury, MN (US); Alan K. Nachtigal, Maplewood, MN (US); Charles J. Hoy, Maple Plain, MN (US); Richard J. Pokorny, Maplewood, MN (US); William J. Hunt, Afton, MN (US); Jason T. Petrin, Woodbury, MN (US); Paul B. Armstrong, St. Paul, MN (US); Suresh Iyer, Woodbury, MN (US)

(21) Appl. No.: 15/537,899

(22) PCT Filed: Dec. 14, 2015
(86) PCT No.: PCT/US2015/065502

§ 371 Date: Jun. 20, 2017

Related U.S. Application Data
(60) Provisional application No. 62/095,516, filed on Dec. 22, 2014.

(51) Int. Cl. C07D 211/44; C07D 401/14; C07D 211/92; C08K 5/3432; C08K 5/3462

(52) U.S. Cl. CPC C07D 211/44; C07D 401/14; C07D 211/92; C08K 5/3462; C08L 2201/08; C08K 5/3432

(57) ABSTRACT

Compounds having hindered amine and oxyalkyl amine light stabilizers can mitigate the adverse effects of actinic radiation, such as visible and ultraviolet light, on polymers and copolymers. Polymers and copolymers derived from such compounds. Articles, such as coated articles and molded articles, containing such polymers or compounds.

### TECHNICAL FIELD

The present disclosure relates to sterically hindered alkyl amine and sterically hindered oxyalkyl amine compounds.

### BACKGROUND

Compounds containing sterically hindered alkyl amines or sterically hindered oxyalkyl amines, and particularly the moiety

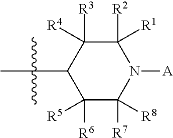

wherein R¹ is alkyl, R² is alkyl, R³ is H or alkyl, R⁴ is H or alkyl, R⁵ is H or alkyl, R⁶ is H or alkyl, R⁷ is alkyl, and R⁸ is alkyl are known in the art. When A is alkyl, such compounds are known as hindered amine light stabilizers, or HALS; when A is oxyalkyl, such compounds are known as NORHALS.

The utility of HALS and NORHALS as radical scavengers and polymer stabilizers and is well recognized in the art, and is described in, for example, the Journal of Macromolecular Science Part A, 35:7, 1327-36 (1998) and The Journal of Macromolecular Science Part A, 38:2, 137-58 (2001), as well as in JP 2001270859, U.S. Pat. No. 4,983,737 (Grant), and U.S. Pat. No. 5,442,071 (Grant). Such compounds are known to protect polymers from adverse effects of actinic radiation, such as visible and ultraviolet light.

### SUMMARY

A compound can have the structure of Formula (I):

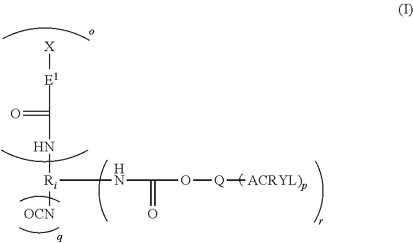

wherein
X is

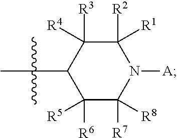

R¹ is alkyl,
R² is alkyl,
R³ is H or alkyl,
R⁴ is H or alkyl,
R⁵ is H or alkyl,
R⁶ is H or alkyl,
R⁷ is alkyl, and
R⁸ is alkyl;
A is alkyl or oxyalkyl;

Rⁱ is a residue of a multi-isocyanate;
E¹ is O or NR′,

R′ is H or C₁ to C₄ alkyl;
each Q is independently a connecting group having a valence of p+1;
each ACRYL is independently an (alk)acryl functional group of the formula OC(O)C(R^{d})═CH₂,

wherein

R^{d} is alkyl or H; and
p is the number of ACRYL groups attached to Q, which is from 1 to 6;
o is the number of

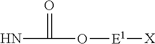

groups covalently bound to Rᵢ, which is from 1 to 8;
r is the number of

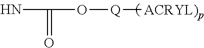

groups covalently bound to Rᵢ, which is from 1 to 8;
q is the number of NCO groups covalently bound to Rᵢ, which is from 0 to 8;
and the sum of q+o+r is from 2 to 10.

In addition to containing hindered alkyl or oxyalkyl amine groups, compounds of Formulas (I) and (II) feature one or more, (alk)acrylate groups, and, in some cases, also feature one or more isocyanate groups. These groups allow the compounds of Formulas (I) and (II) to be incorporated into the backbone of polymers, such as polyurethanes, polyolefins, polyacrylates, polystyrenes, and the like.

### DETAILED DESCRIPTION

Throughout this disclosure, singular forms such as “a,” “an,” and “the” are often used for convenience; however, it should be understood that the singular forms are meant to include the plural unless the singular alone is explicitly specified or is clearly indicated by the context.

Some terms used in this application have special meanings, as defined herein. All other terms will be known to the skilled artisan, and are to be afforded the meaning that a person of skill in the art at the time of the invention would have given them.

“Independently,” when used in reference to the identity of one or more variable elements, means that each occurrence of any of the variable elements may have the same or different identity, within the specified limitations, regardless of the identity of any other occurrence of the reference element. Thus, if there are two occurrences of element “X,” and element X can be independently selected from identity Y or identity Z, each of the two occurrences of X can be either Y or Z, in any combination (e.g., YY, YZ, ZY, or ZZ).

“Alkyl” refers to a saturated hydrocarbon radical. Many alkyl groups are from C₁ to C₃₀. Some alkyl groups can be C₁ or greater, such as C₂ or greater, C₄ or greater, C₆ or greater, or C₈ or greater. Some alkyl groups can be C₂₂ or smaller, C₁₆ or smaller, C₁₂ or smaller, C₈ or smaller, or C₄ or smaller. Unless otherwise indicated, any alkyl group can independently be linear, branched, cyclic, or a combination thereof (e.g., a cyclic alkyl can also have a linear or branched component.) Exemplary alkyl groups include methyl, ethyl, propyl, isopropyl, n-butyl, t-butyl, sec-butyl, iso-butyl, 2-ethyl hexyl, iso-octyl, n-octyl, dodecyl, hexadecyl, behenyl, and the like.

“Oxyalkyl” refers to a monovalent radical having the formula O-alkyl, which can be referred to as an alkoxy group. The alkyl portion of the oxyalkyl can be any alkyl, such as those discussed above with reference to the definition of the term alkyl. Oxyalkyl can be written using standard preffixes to indicate the number of carbon atoms in the alkyl portion of the oxyalkyl. For example, oxymethyl is an oxyalkyl wherein the alkyl portion has one carbon, oxyethyl is an oxyalkyl wherein the alkyl portion has two carbons, etc. Oxyoctyl is an exemplary oxyalkyl that is often used in the compounds described herein.

“Alkylene” refers to an aliphatic hydrocarbon diradical (I.e. a divalent radical). Many alkylene diradicals are from C₁ to C₃₀. Alkylene diradicals can be C₁ or greater, C₂ or greater, C₃ or greater, C₄ or greater, C₆ or greater, or C₈ or greater. Alkylene diradicals can be C₂₂ or smaller, C₁₆ or smaller, C₁₂ or smaller, C₁₀ or smaller, or C₈ or smaller. Unless otherwise indicated, any alkylene can be linear, branched or cyclic or a combination thereof (e.g., having both a cyclic component and a linear component.) Exemplary alkylene groups include methylene, ethylene, propyl, isopropylene, n-butylene, t-butylene, sec-butylene, iso-butylene, 2-ethylhexylene, iso-octylene, dodecylene, hexadecylene, behenylene, and the like. In this application, hexylene is often used as an alkylene.

“Isocyanate” refers to a molecule comprising at least one isocyanato group, which is a —NCO.

A “multi-isocyanate” is an isocyanate molecule comprising at least two isocyanate radicals.

A polymer or copolymer is “derived from” a reference compound when the backbone of the polymer or copolymer contains a polymerized form of the reference compound.

A “hydrocarbon polyradical” as used herein is an aliphatic multivalent radical containing only carbon and hydrogen atoms. Hydrocarbon polyradicals can be C₁ or greater, C₂ or greater, C₃ or greater, C₄ or greater, C₆ or greater, or C₈ or greater. Hydrocarbon polyradicals can be C₂₂ or smaller, C₁₆ or smaller, C₁₂ or smaller, C₁₀ or smaller, or C₈ or smaller. In many embodiments, the polyradicals are divalent or trivalent.

Compounds of Formula (I) feature E¹ that is either O or NHR′ with R′ being H or C₁ to C₄ alkyl. When each E is O, the compound of Formula (I) is a compound of Formula (II). When each E¹ is NHR′, the compound of Formula (I) is a compound of Formula (IIa).

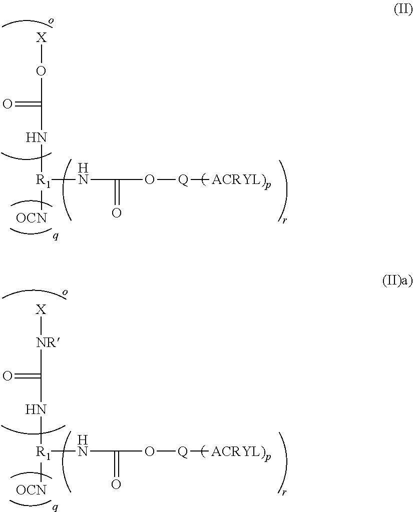

Compounds of Formulas (I), (II), or (IIa) can be synthesized from compounds of Formula (III).

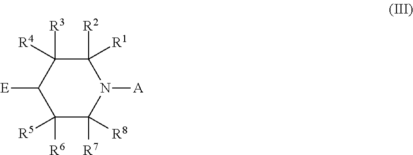

In the compound of Formula (III), R¹ through R⁸, and A have the same meaning as in the compound of Formula (I), and E is OH or NHR′, wherein R′ has the same meaning as in the compound of Formula (I).

In any compound of Formula (III), R¹, R², R⁷, and R⁸ can be independently any suitable alkyl. R¹, R², R⁷, and R⁸ can be the same or different. Typical alkyls for any of R¹, R², R⁷, and R⁸ include C₁ or greater, C₂ or greater, C₃ or greater, C₄ or greater, C₆ or greater, C₈ or greater, or C₁₂ or greater. Other typical alkyls that can be used as one or more of R¹, R², R⁷, and R⁸ include C₁₆ or less, C₁₂ or less, C₈ or less, C₆ or less, C₄ or less, C₃ or less, or C₂ or less. In many cases, each of R¹, R², R⁷, and R⁸ are methyl.

R³, R⁴, R⁵, and R⁶ can be independently H or alkyl. When one or more of R³, R⁴, R⁵, and R⁶ is alkyl, the alkyl is typically C₁ or greater, C₂ or greater, C₃ or greater, C₄ or greater, C₆ or greater, C₈ or greater, or C₁₂ or greater. Such alkyl is often C₁₆ or less, C₁₂ or less, C₈ or less, C₆ or less, C₄ or less, C₃ or less, or C₂ or less. In many cases, one or more of R³, R⁴, R⁵, and R⁶ is H. Most commonly, each of R³, R⁴, R⁵, and R⁶ are H.

The identity of each of R¹ through R⁸ in a compound of Formula (III) is carried over into compounds of Formula (I) that are synthesized from that compound of Formula (III). Thus, the identity of each of R¹ through R⁸ in any compound of Formula (I) will depend on, and be the same as, the identity of the R¹ through R⁸ in the compound or compounds of Formula (III) used as a starting material.

In some cases, E in the compound of Formula (III) is hydroxy. When such compound is employed as a starting material, the resulting compound of Formula (I) will be a compound of Formula (II). Similarly, when E is NR′, the resulting compound of Formula (I) will be a compound of Formula (IIa). A can be either alkyl or oxyalkyl. When A is alkyl, then the compound of Formula (III) is a compound of, for example, Formula (IIIa). When A is oxyalkyl, then the compound of Formula (III) is a compound of, for example, Formula (IIIb).

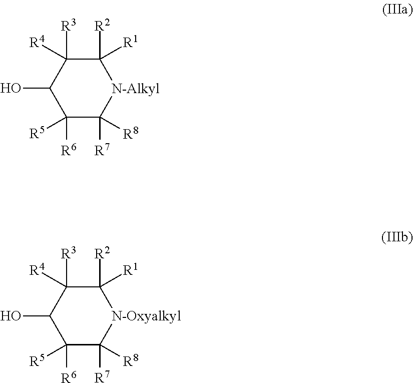

The alkyl in the compound of Formula (IIIa) can be any suitable alkyl. The alkyl can be linear, branched, cyclic, or a combination thereof (e.g., a cyclic alkyl that also has a linear component.) Typical alkyls are C₁ or greater, C₂ or greater, C₃ or greater, C₄ or greater, C₆ or greater, C₈ or greater, or C₁₂ or greater. Many alkyls are C₁₆ or less, C₁₂ or less, C₈ or less, C₆ or less, C₄ or less, C₃ or less, or C₂ or less. In many cases, the alkyl is C₁ to C₄ alkyl. Methyl is most common.

Most commonly compounds of Formula (Ma) feature R¹, R², R⁷, and R⁸ that are methyl, R³, R⁴, R⁵, and R⁶ that are H. In such cases, the compound of Formula (III) is a compound of Formula (IIIa1). The alkyl, which is connected to the nitrogen in the ring, in the compound of Formulas (IIIa) and (IIIa1) is most often methyl. In such cases, the compound of Formula (IIIa1) is a compound of Formula (IIIa2).

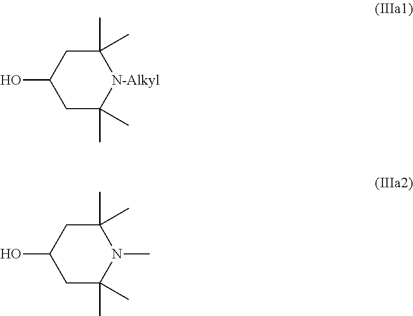

Most commonly compounds of Formula (IIIb) feature R¹, R², R⁷, and R⁸ that are methyl, and R³, R⁴, R⁵, and R⁶ that are H. In such cases, the compound of Formula (IIIb) is a compound of Formula (IIIb1).

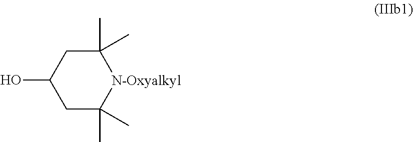

The oxyalkyl in the compound of Formula (IIIb) or (IIIb1), which is connected to the nitrogen in the ring, can be any suitable oxyalkyl. The oxyalkyl can be linear, branched, cyclic, or a combination thereof (e.g., a cyclic oxyalkyl can also have a linear component.) Typical oxyalkyls are C₁ or greater, C₂ or greater, C₃ or greater, C₄ or greater, C₆ or greater, C₈ or greater, C₁₂ or greater, C₁₆ or greater, or C₂₂ or greater. Many oxyalkyls are C₂₆ or less, C₂₂ or less, C₁₈ or less, C₁₆ or less, C₁₂ or less, C₈ or less, C₆ or less, C₄ or less, C₃ or less, or C₂ or less. C₈ oxyalkyl is often used. In most cases, compounds of Formula (IIIb) or (IIIb1) contain a mixture of linear and branched isomers of the oxyalkyl group. This effect has been noted in documents that describe the preparation of such compounds, such as Schoening et. al. (J. Org. Chem. 2009, 74, 1567-1573), U.S. Pat. No. 4,983,737, U.S. Pat. No. 5,286,865, U.S. Pat. No. 5,442,071 and US2010/0249401. Of the C₈ isomers, which are collectively known as oxyoctyl, branched isomers tend to occur more often than the linear isomer. When the oxyalkyl in the compound of Formula (IIIb1) is oxyoctyl, the compound of Formula (IIIb1) is a compound of Formula (IIIb2).

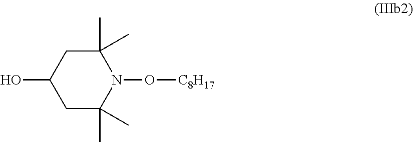

In other cases, E in the compound Formula (III) can be NHR′. When such compounds are employed as starting materials, the resulting compounds of Formula (I) will be compounds of Formula (IIa). A can be alkyl or oxyalkyl. When A is alkyl, the compound of Formula (III) is a compound of Formula (IV). When A is oxyalkyl, the compound of Formula (III) is a compound of Formula (IVa).

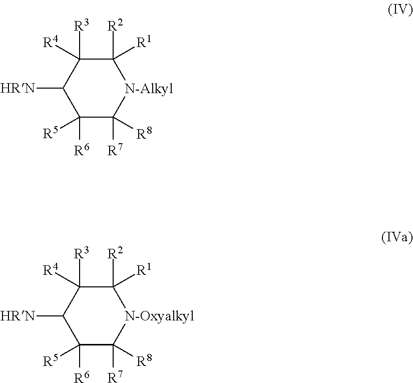

In compounds of Formula (IV) and (IVa), the identity of each of R¹ through R⁸ is the same as in the compound of Formula (III). Most commonly, compounds of Formula (IV) feature R¹, R², R⁷, and R⁸ that are methyl, and R³, R⁴, R⁵, and R⁶ that are H. In such cases, the compound of Formula (IV) is a compound of Formula (IV1)

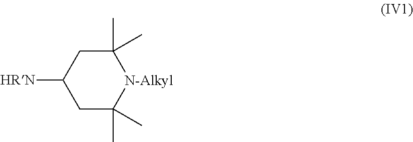

R′ in the compound of Formula (IV) or (IV1) can be H or any C₁ to C₄ alkyl. When R′ is alkyl, methyl and ethyl are most common. Typically, R′ is H, in which case the compound of Formula (IV1) is a compound of Formula (IV2)

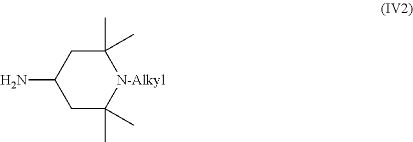

The alkyl, which is connected to the nitrogen in the ring, in the compound of Formula (IV), (IV1), or (IV2) can be any suitable alkyl, such as those discussed above with respect to the compound of Formulas (IIIa). Methyl is most common, in which case the compound of Formula (IV2) is a compound of Formula (IV3).

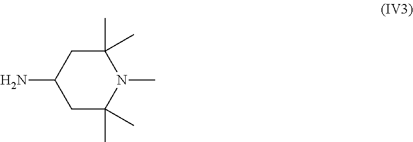

In the compound of Formula (IVa), the identity of each of R¹ through R⁸ is the same as in the compound of Formula (III). Most commonly, compounds of Formula (IVa) feature R¹, R², R⁷, and R⁸ that are methyl, R³, R⁴, R⁵, and R⁶ that are H. In such cases, the compound of Formula (IVa) is a compound of Formula (IVa1).

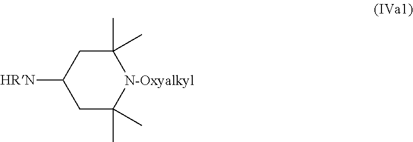

R′ in the compound of Formula (IVa) or (IVal) can be H or any C₁ to C₄ alkyl. When R′ is alkyl, methyl and ethyl are most common. Typically, R′ is H, in which case the compound of Formula (IVa1) is a compound of Formula (IVa2)

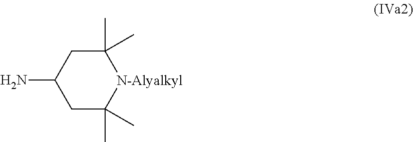

In the compounds of Formulas (IVa), (IVa1), and (IVa2) the oxyalkyl, which is connected to the nitrogen in the ring, can be any suitable oxyalkyl, such as those discussed above with respect to the compound of Formula (IIIb). Oxyoctyl is most common, in which case the compound of Formula (IVa2) is a compound of Formula (IVa3).

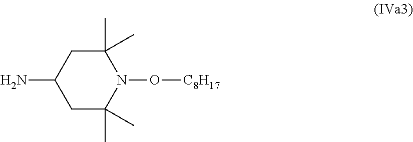

The various compounds of Formula (III) discussed herein can be used in the synthesis of compounds of Formulas (I), (II), or (IIa). For example, compounds of Formula (IIa) can be used as starting materials for compounds of Formula (I) wherein A is alkyl and E¹ is O, which are also compounds of Formula (II). Typically, compounds of Formula (IIIa2) are used for this purpose. Compounds of Formula (IIIa) can also be used as starting materials for compounds of Formula (II) wherein A is alkyl and L₂ is O. Compounds of Formula (IIIa) are sometimes known as 2,2,6,6-tetraalkyl-4-hydroxy N-alkylpiperidines, and are commercially available. Exemplary compounds of Formula (Ma), (IIIa1), and (IIIa2) can be obtained from TCI America (OR, USA), for example, under the trade designation PMHP.

As another example, compounds of Formula (IIIb), (IIIb1), and (IIIb2) can be used as starting materials for compounds of Formula (I) wherein A is oxyalkyl and E¹ is O, which are also compounds of Formula (II). Compounds of Formula (IIIb) are sometimes known as alkylated N-oxyalkyl 4-hydroxy piperidines, and can be prepared from commercially available bis(alkyated N-oxyalkyl-4-piperidyl) esters of alkylene diacids as shown in Reaction Scheme 1. Exemplary bis(alkylated N-oxyalkyl-4-piperidyl) esters of alkylene diacids can be obtained from BASF (NJ, USA), for example, under the trade designation TINUVIN 123.

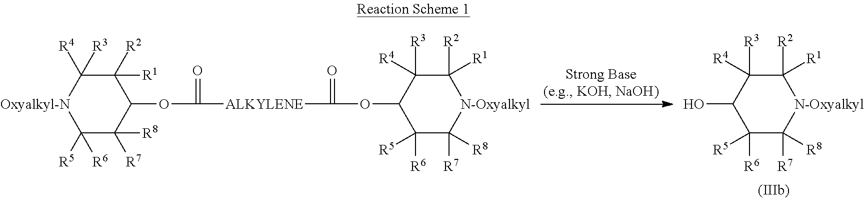

As shown in Reaction Scheme 1, treating a bis(alkylated N-oxyalkyl-4-piperidyl) ester of alkylene diacids with a strong Arrhenius base, for example an alkali metal hydroxide such as potassium hydroxide or sodium hydroxide, hydrolyzes the esters to form an alkylated N-oxyalkyl 4-hydroxy piperidine. This reaction can take place under any suitable conditions for hydrolyzing diacids. The reaction often takes place in the presence of one or more inert diluents. The one or more inert diluents are typically used to dissolve or disperse the strong Arrhenius base, the bis(alkylated N-oxyalkyl-4-piperidyl) esters of alkylene diacids, or both. Typical inert diluents include alcohols, such as methanol, ethanol, or isopropanol. The reaction can be promoted by heating. When one or more alcohols are used as the inert diluents, heating can involve refluxing the one or more alcohols. The starting material of Reaction Scheme 1 is often a bis(2,2,6,6-tetramethyl-N-oxyalkyl-4-piperidyl) ester, in which case the product of Reaction Scheme 1 is the compound of Formula (IIIb2).

Compounds of Formula (IV), including compounds of Formula (IV1), (IV2), and (IV3), can be used as starting materials for compounds of Formula (I) wherein A is alkyl and E¹ is NR′. Compounds of Formula (IVa), including compounds of Formulas (IVa1), (IVa2), and (IVa3), can be used as starting materials for compounds of Formula (I) wherein A is oxyalkyl and E¹ is NR′.

Compounds of Formula (IV) and (IVa) wherein R′ is H are compounds of Formula (V) and (Va), respectively. Such compounds can be synthesized from compounds of Formula (IIIa) or (IIIb), respectively, as shown in Reaction Scheme 2 and Reaction Scheme 3. First, compounds of Formulas (III) or (IIIa) can be converted to ketone intermediates of Formula (IIIb) or (IIIc) by Swern oxidation of the hydroxy group in the compounds of Formula (III) with oxalyl chloride and dimethyl sulfoxide (DMSO) followed by quenching with triethylamine. The ketone intermediates of Formula (IIIc) or (IIId) can then be converted to compounds of Formula (IV) or (IVa), respectively, by reductive amination. Reductive amination can be accomplished by any suitable procedure, such as treatment with sodium cyanoborohydride and ammonia or an amine, which is typically a ammonia or a protonated amine, that is, an ammonium salt, such as ammonium acetate.

The nature of the amine used in the reductive amination reaction determines the identity of R′ in the compound of Formula (IV1) or (IVa). Thus, if ammonium is used, as in Reaction Scheme 2, R′ in the resulting compound of Formula IV1 or IVa is H.

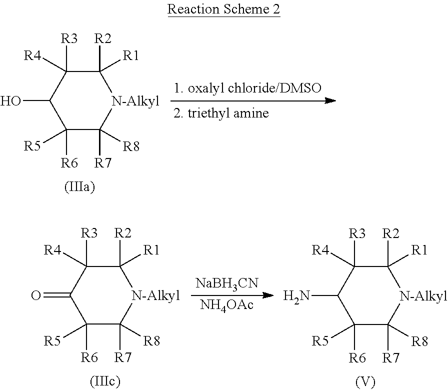

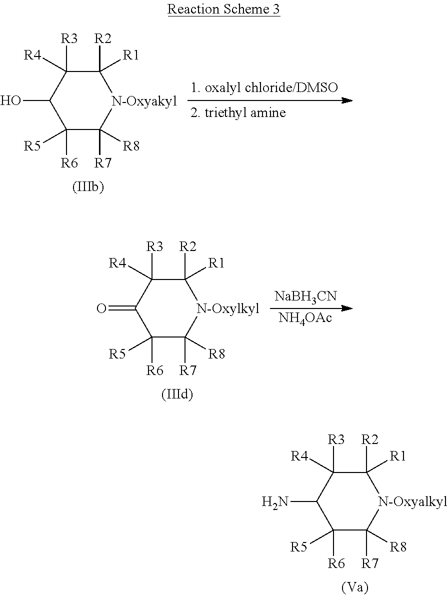

Conditions for Swern oxidation of alcohols to ketones are known to people of ordinary skill in the art, and have been disclosed, for example, in “Oxidation of alcohols by ‘activated’ dimethyl sulfoxide. A preparative, steric and mechanistic study” Tetrahedron 34 (11) 1978 (Omura et al.), and “Oxidation of alcohols by activated dimethyl sulfoxide and related reactions: An update” Synthesis (10); 857-70 (Tidwell et al.) Conditions for reductive amination of carbonyls with sodium cyannoborohydride are also known to people of ordinary skill in the art, and have been disclosed, for example, in “Reductive amination with sodium cyanoborohydride: N,N-dimethylcyclohexylamine”, Org. Synth. Coll. Vol. 6: 499, 1988 (Borch), and “Cyanohydriodoborate anion as a selective reducing agent”, J. Am. Chem. Soc. 95 (12), 1971 (Borch et al.)

As discussed above, one method to provide compounds of Formula (IV) or (IVa) wherein R′ is C₁ to C₄ alkyl is the use of a primary alkyl amine compound in the reductive amination reaction. As an alternative, compounds of Formulas (V) or (IV) can be alkylated by reaction of the primary amine with a compound of Formula (VI), as shown in Reaction Schemes 4 and 5. The resulting compounds wherein R′ is C₁ to C4 alkyl are compounds of Formula (IVb) or (IVc). The chemical structure of compounds of Formula (IVb) and (IVc) is identical whether such compounds are made by reductive amination with a primary alkyl amine in a process similar to Reaction Scheme 2 or 3 or by alkylation as shown in Reaction Schemes 4 and 5.

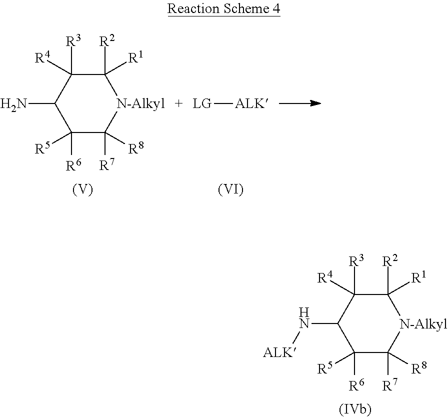

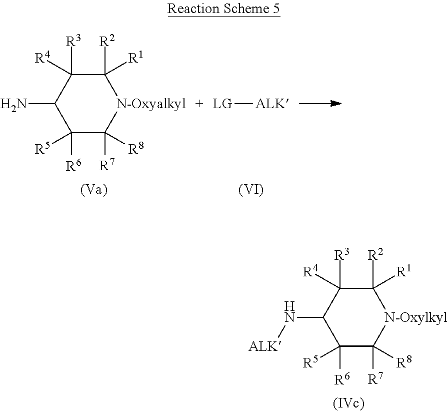

In the compound of Formulas (VI), ALK′ is C₁ to C₄ alkyl and LG is a leaving group. Any suitable leaving group that can be used, so long as the compound of Formula (VI) is reactive with the exocyclic amine of a compound of Formulas (V) or (Va). Suitable leaving groups include halide, such as chloride, bromide, and iodide, mesylate, tosylate, and the like. Likewise, ALK′ any suitable C₁ to C₄ alkyl can be used. Typical examples of C₁ to C₄ alkyl include methyl, ethyl, n-propyl, iso-propyl, and n-butyl. Methyl and ethyl are most common.

The ALK′ moiety in the compounds of Formulas (IVb) and (IVc) comes from the ALK′ group of compounds of Formula (VI), and is defined in the same way as that in compounds of Formula (VI).

The reaction shown in Reaction Schemes 4 and 5 can take place under any reaction conditions suitable for alkylation of a primary amine. Typically, the compound of Formula (V) or (Va) is first dissolved or dispersed in one or more inert diluents that do not undergo a chemical reaction under the alkylation conditions. Common inert diluents include aromatics such as benzene, toluene, and xylenes, ethers such as diethyl ether and tetrahydrofuran, as well as hydrocarbons such as hexanes. The compound of Formula (VI) can be added to the compound of Formula (V) or (Va) and the inert diluents in any suitable manner. For example, the compound of Formula (VI) can be added to the compound of Formula (V) or (Va) and the one or more inert diluents dropwise with a syringe. The reaction often takes place at ambient temperatures, but it can be facilitated by heating if necessary.

Any compound of Formulas (III), (IV), or (IVa), such as those discussed herein, can be converted into a compound of Formula (I). For example, a compound of Formula (I) can be formed by reacting any compound of Formula (III), (IV), or (IVa) with a multi isocyanate. The multi-isocyanate typically has between 2 and 10 isocyanate groups. Multi-isocyanates have two or three isocyanate groups are most common.

Exemplary multi isocyanates include compounds of Formula (VII). In compounds of Formula (VII), G is alkylene. The identity of G is carried forward into the resulting compound of Formula (I), (II), or (IIa) that are produced from compounds of Formula (VII). As such, the identity of Gin any compound of Formula (I) or (II) will depend on, and be the same as, the identities of G discussed here with respect to compounds of Formula (VII).

G can be any suitable alkylene. In many cases, G is C₁ or greater, C₂ or greater, C₃ or greater, C₄ or greater, C₆ or greater, C₈ or greater, or C₁₂ or greater. G is often C₁₆ or less, C₁₂ or less, C₈ or less, or C₆ or less. G is most often linear, but when G is C₃ or greater it is possible for G to be linear, branched, cyclic, or a combination thereof (e.g., alkylene having a cyclic component and a linear component). One common G is linear C₆ alkylene. The value of n is typically between 0 and 8.

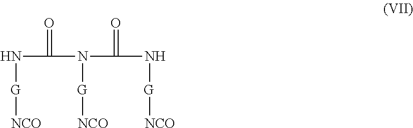

Exemplary compounds of Formula (VII) are commercially available. Exemplary compounds of Formula (VII) can be obtained from Bayer Polymers LLC (Pittsburgh, USA). One such compound is obtainable under the trade designation DESMODUR N100. In such compounds, G is typically hexylene and the compound of Formula (VII) can be represented by Formula (VIIa).

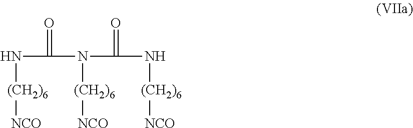

Many of the multifunctional isocyanates of greater than 2 functionality, including those of Formulas (VII) and (VIIa), exist as a distribution of materials. For instance, hexamethylene diisocyanate based isocyanate oligomers such as biuret multi-isocyanates (for instance those available under the trade designation DESMODUR N100) exist as a mixture of hexamethylene diisocyanate, hexamethylene diisocyanate biuret trimers, hexamethylene diisocyanate biuret pentamers, hexamethylene diisocyanate biuret heptamers, and so on. The same is true for hexamethylene diisocyanate based isocyanurate multi-isocyanates (for instance those available under the trade designation DESMODUR N3300). Biuret and isocyanurate multi-isocyanates may be based on other diisocyanates such as isophorone diisocyanate, or tolylene diisocyanate. In drawing structures, only the trimers of these materials are shown for simplicity, since the trimmers are believed to be the most prevalent structures in the commercial products.

Compounds of Formula (VII), such as those discussed herein, can react with compounds of any compound of Formula (III), including compounds of Formulas (IIIa), including (IIIa1) or (IIIa2), (Mb), including (IIIb1) or (IIIb2), (IV), including (IV1), (IV2), or (IV3), or (IVa), including (IVa1), (IVa2), or (IVa3), to form one or more compounds of Formula (I). This reaction is shown in Reaction Scheme 6.

Compounds of Formulas (VIII), (VIIIa), (VIIIb), (VIIIc), and (VIId) are all compounds of Formula (I) wherein Rᵢ is the HN(G)C(O)N(G)C(O)NH(G) residue of the multi isocyanate of Formula (VIIa). Thus, the identity of G in such Rᵢ is identical to the identity of G in the compounds of Formula (VII) or (VIIa), and is often alkyl, such as C₁ or greater, C₂ or greater, C₃ or greater, C₄ or greater, C₆ or greater, C₈ or greater, or C₁₂ or greater. G is often C₁₆ or less, C₁₂ or less, C₈ or less, or C₆ or less. Hexyl is common. The identity of other variable elements, such as A and E¹, is identical to that of the compound of Formula (III) that is used in Reaction Scheme 6.

Compounds of Formulas (VIII) and (VIIIa) are precursors of Formula (I). The identity of A in each of the compounds of Formulas (VIII), (VIIIa), (VIIIb), (VIIIc), and (VIId) is identical to the identity of A in the compound of Formula (III) from which they are obtained. The identity of E¹ in compounds of Formulas (VIII), (VIIIa), (VIIIb), (VIIIc), and (VIId) depends on the identity of E in the compound of Formula (III). When E in the compound of Formula (III) is NR′H, then E¹ in the compounds of Formulas (VIII), (VIIIa), (VIIIb), (VIIIc), and (VIId) is NR′, wherein R′ is the same R′ as in the compound of Formula (III). When E in the composition of Formula (III) is hydroxy, then E¹ in the compounds of Formulas (VIII), (VIIIa), (VIIIb), (VIIIc), and (VIId) is 0.

The reaction of a compound of Formula (III) with a compound of Formula (VIIa) provides a plurality of products, because group E the compound of Formula (III) can react with one or more of the isocyanate moieties on the compound of Formula (VII) or (VIIa). The number of isocyanate groups that react can be influenced by changing the stoichiometric ratio of the compound of Formula (III) and the compound of Formula (VIIa). Using one equivalent or less of the compound of Formula (III), with respect to the number of isocyanate moieties in the compound of Formula (VII) or (VIIa), favors the formation of the mono-substituted compounds of Formulas (VIII) and (VIIIa). Using three equivalents or more of the compound of Formula (III), with respect to the number of isocyanate moieties in the compound of Formula (VII) or (VIIa), favors the formation of the tri-substituted compound of Formula (VIIIc). In most cases, however, the result of the reaction of a compound of Formula (III) with a compound of Formula (VIIa) is a mixture of compounds of Formulas (VIII), (VIIIa), (VIIIb), and (VIIIc).

Each of the compounds of Formulas (VIII), (VIIIa), (VIIIb), and (VIIIc) are precursors of Formula (I). Compounds of Formulas (VIII), (VIIIa), or (VIIIb) can undergo further chemical reactions to form compounds of Formula (I) by procedures discussed herein. Compounds of Formula (VIIIc) have no remaining reactive isocyanate groups, and therefore cannot undergo such further chemical reactions to form a compound of Formula (I).

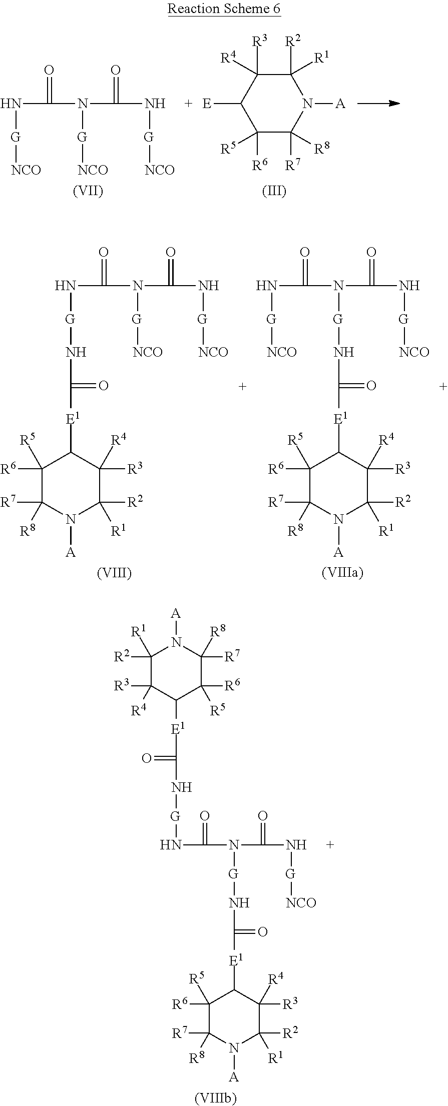

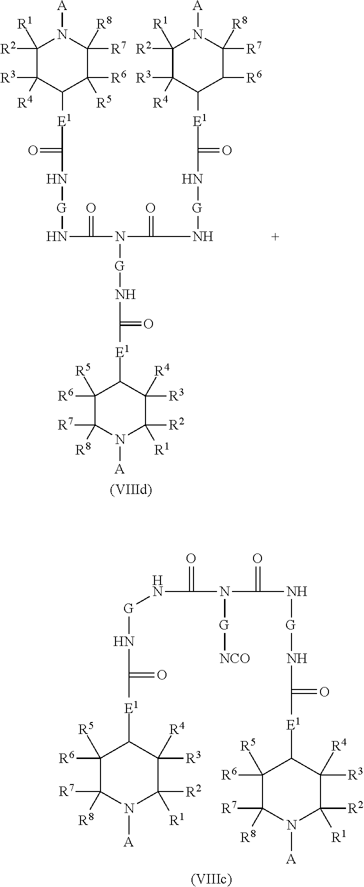

In many cases, the compound of Formulas (VIII), (VIIIa), (VIIIb), (VIIIc), and (VIIId) feature R¹, R², R⁷, and R⁸ that are methyl, R³, R⁴, R⁵, R⁶ that are H, as well as an E¹ that is O. In other cases, the compounds of Formulas (VIII), (VIIIa), (VIIIb), (VIIIe), and (VIIId) feature R¹, R², R⁷, and R⁸ that are methyl, R³, R⁴, R⁵, R⁶ that are H, as well as an E¹ that is NR′, typically NH.

G in such compounds is typically hydrocarbon polyradical, particularly C₁ to C₁₂ or C₁ to C₆ hydrocarbon polyradical. The hydrocarbon polyradical is often alkylene, in which case the alkylene is often C₁ to C₁₂ or C₁ to C₆ alkylene. Hexylene is most common.

In such compounds, A can be alkyl or oxyalkyl. When alkyl is employed, the alkyl is typically C₁ to C₁₂ alkyl, such as C₁ to C₆ alkyl. Methyl is most common. When oxyalkyl is employed, the oxyalkyl is typically C₁ to C₁₂ oxyalkyl. Oxyoctyl is most common.

One or more of the isocyanate moieties in compounds of any of Formulas (VIII), (VIIIa), or (VIIIb) can be converted to (alkyl)acrylate-containing compounds of Formula (I). Such conversion can be accomplished by any suitable chemical transformation. One suitable transformation is a reaction with a hydroxy-containing acrylate or multi-acrylate, such as a compound of Formula (IX).

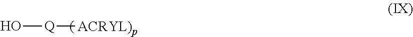

In Formula (IX), Q is a connecting group, each ACRYL is independently a (meth)acryl functional group of the formula OC(O)C(R^{d})═CH₂, and p is the number of (meth)acryl functional groups attached to Q, which be from 1 to 6. Q can be any suitable connecting group, such as hydrocarbon polyradical, alkylene, alkenylene, alkynylene, alkyleneoxyalkylene, alkyleneneaminoalkylene, and the like. For example Q can be a linear, branched, or cycle-containing connecting group. Q can include a covalent bond, alkylene, arylene, or aralkylene. Q can optionally include heteroatoms, most often one or more of O, N and S. Q can also optionally include heteroatom containing functional groups, such as carbonyl, sulfonyl, or both.

Q is most commonly hydrocarbon polyradical or alkylene. Hydrocarbon polyradical is most common when p is greater than 1. Common hydrocarbon polyradicals include C₁ to C₁₂ hydrocarbon polyradical, such as C₁ to C₆ hydrocarbon polyradical. Q is typically alkylene when p is 1. Common alkylenes include C₁ to C₁₂ alkylene, such as C₁ to C₆ alkylene, for example ethylene, ethylene, propylene, butylene, and the like. In most cases, p is 1 to 3, with 1 and 3 being most common.

The identity of p, Q, and ACRYL, including R^{d}, in compounds of Formula (IX) carries over into any compound that is prepared from a compound of Formula (IX). Thus, any compound that can be prepared from a compound of Formula (IX), such as any compound of Formula (I) having an r of 1 or greater, will have a p, Q, and ACRYL, including R^{d}, with identities that are the same as that discussed above with respect to compounds of Formula (IX).

Many compounds of Formula (IX) are commercially available. Exemplary compounds of Formula (IX) wherein p is 1 include of hydroxyalkyl (meth)acrylates, such as 2-hydroxyethyl acrylate, methyl 2-(2-hydroxy-l-methylethyl)acrylate, and methyl 2-(2-hydroxy-1-phenylethyl)acrylate, all of which are available from Sigma-Aldrich (Milwaukee, USA). Exemplary compounds of Formula (IX) wherein p is greater than 1 include pentaerythritol triacrylate which is available from Sartomer Company (Exton, Pa. USA) under the trade designation SR444C, and 3-(acryloxy)-2-hydroxypropyl methacrylate (CAS number 1709-71-3) available from Sigma-Aldrich (Milwaukee, Wis. USA).

Compounds of Formula (IX) can react with compounds of Formulas (VIII), (VIIIa), or (VIIIb) according to Reaction Schemes 7, 8, and 9, respectively. The reaction can be conducted under any suitable conditions for reaction of a hydroxy with an isocyanate. In many cases, the reaction can be conducted at ambient temperature by stirring the compound of Formula (IX) with a compound of Formula (VIII), (VIIIa), or (VIIIb) in one or more inert diluents. Typical inert diluents do not undergo chemical reactions under the reaction conditions, and include aromatics such as benzene and toluene, ethers such as diethyl ether and tetrahydrofuran, and chlorinated diluents such as dichloromethane and chloroform. The reaction can often take place at ambient temperatures; however, the reaction can be facilitated by heating, for example, to approximately 60° C.

Reaction Scheme 7 shows the reaction of a compound of Formula (VIII) with a compound of Formula (IX). This reaction can be carried out under the same reaction conditions discussed above with respect to the reaction of a compound of Formula (VIIa) with a compound of Formula (III). The reaction results in a mixture of products, which are compounds of Formulas (X), (Xa), and (Xb). Compounds of Formula (X) and (Xa) feature one O-Q-(ACRYL)ₚ group and one isocyanate group per molecule, that is, in such compounds q is 1 and r is 1. Compounds of Formula (Xb) feature two O-Q-(ACRYL)ₚ groups per molecule, that is, q is 0 and r is 2. Some control over the relative amount of the reaction products can be achieved by varying the amount of the compound of Formula (IX) that is used in the reaction. Using less than one equivalent of the compound of Formula (IX) will favor the formation of compounds of Formulas (X) and (Xa), whereas using more than two equivalent will favor the formation of the compound of Formula (Xb).

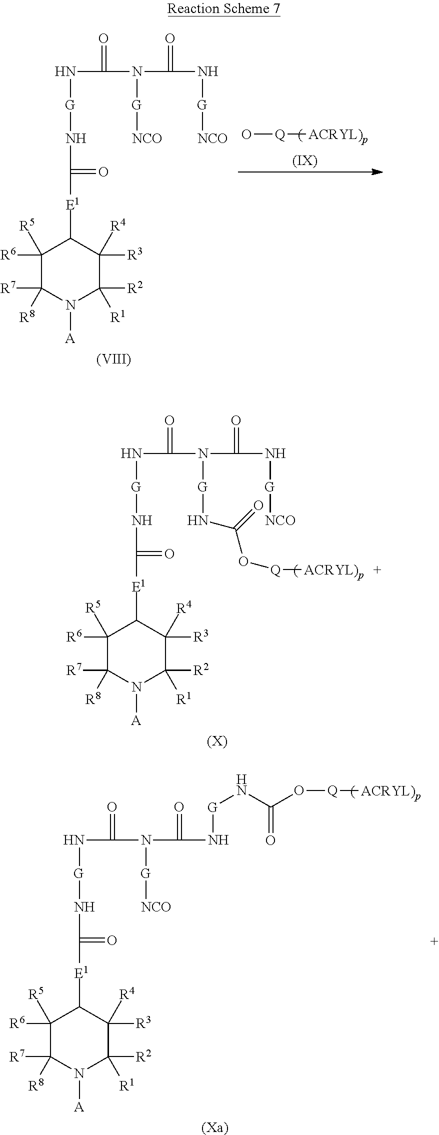

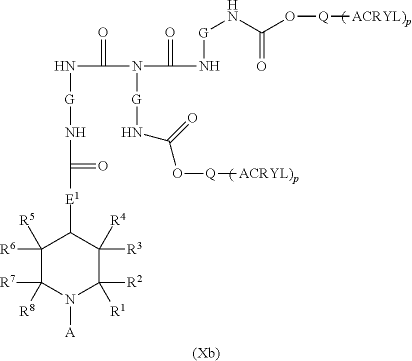

In compounds of Formulas (X), (Xa), and (Xb), the identity of A, le through R⁸, E¹, and each G is carried over from the compound of Formula (VIII). Thus, the identity of any of these elements is the same as that described above with respect to Formula (VIII). Similarly, the identity of Q is carried over from the compound of Formula (IX), and is therefore the same as that described above with respect to the compound of Formula (IX).

ACRYL in these compounds typically features an R^{d} that is methyl or H.

In many cases, compounds of Formulas (X), (Xa), and (Xb) features R¹, R², R⁷, and R⁸ that are methyl, R³, R⁴, R⁵, R⁶ that are H. Such compounds are compounds of Formulas (X1), (Xa1), and (Xb1), respectively.

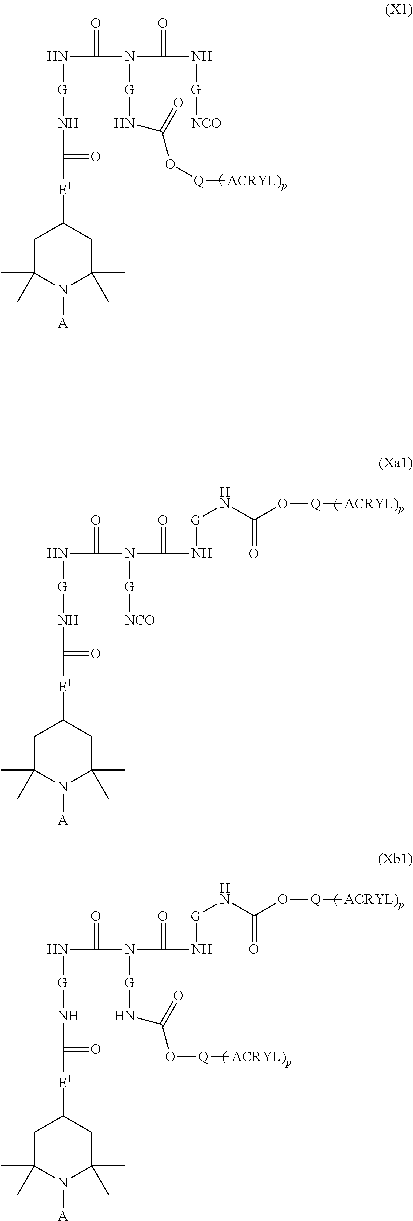

When E¹ in the compounds of Formulas (X1), (Xa1), and (Xb1) is O, then those compounds are compounds of Formulas (X2), (Xa2), and (Xb2), respectively.

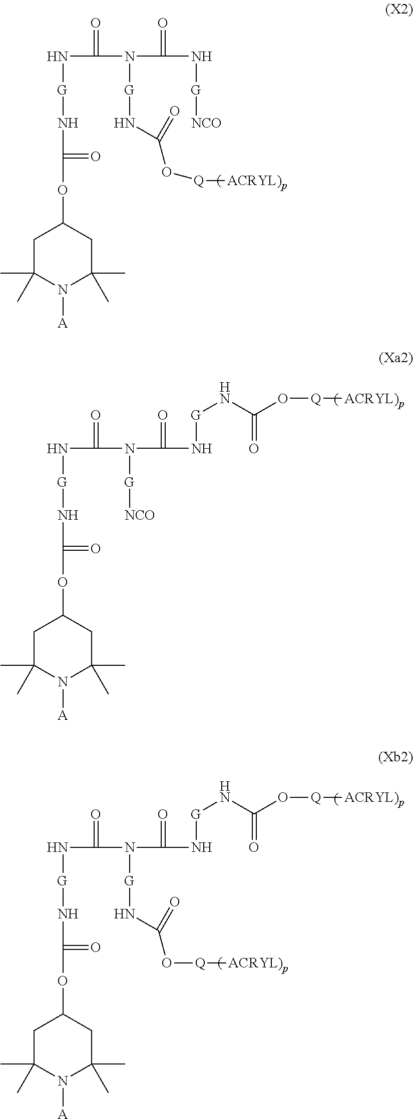

When E¹ in the compounds of Formulas (X1), (Xa1), and (Xb1) is NH, then those compounds are compounds of Formulas (X3), (Xa3), and (Xb3), respectively.

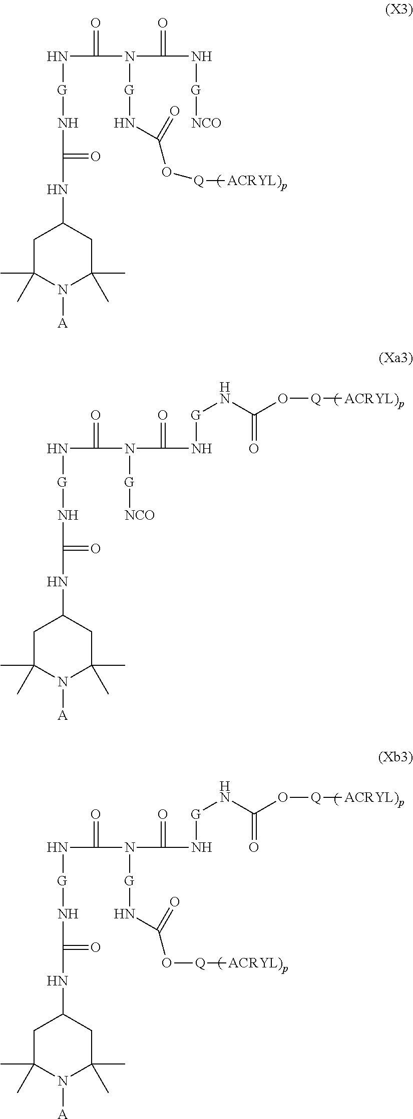

In any of the compounds of Formulas (X2), (Xa2), (Xb3), (X3), (Xa3), and (Xb3), Q is most commonly hydrocarbon polyradical, particularly C₁ to C₁₂ or C₁ to C₆ hydrocarbon polyradical. The hydrocarbon polyradical is often alkylene, in which case the alkylene is often C₁ to C₁₂ or C₁ to C₆ alkylene. Similarly, G is alkylene, most commonly C₁ to C₁₂ alkylene, such as C₁ to C₆ alkylene. Hexylene is most common.

ACRYL in these compounds typically features an R^{d} that is methyl or H.

Reaction Scheme 8 shows the reaction of a compound of Formula (VIIIa) with a compound of Formula (IX). The products of this reaction are compounds of Formulas (XI), (XIa), and (XIb).

Compounds of Formulas (XI), (XIa), and (XIb) are compounds of Formula (I) wherein o is 1. In compounds of Formulas (XI) and (XIa), q is 1 and r is 1. In the compound of Formula (XIb), q is 0 and r is 2. Some control over the relative amount of the reaction products can be achieved by varying the amount of the compound of Formula (IX) that is used in the reaction. Using one equivalent or less of the compound of Formula (IX) favors the formation of compounds of Formulas (XI) and (XIa), whereas using two or more equivalents of the compound of Formula (IX) favors formation of compounds of Formula (XIb).

In compounds of Formulas (XI), (XIa), and (XIb), the identity of E¹, A, R¹ through R⁸, and each G is carried over from the compound of Formula (VIII). Thus, the identity of any of these elements is the same as that described above with respect to Formula (VIII).

The identity of Q is carried over from the compound of Formula (IX), and is therefore the same as that described above with respect to the compound of Formula (IX).

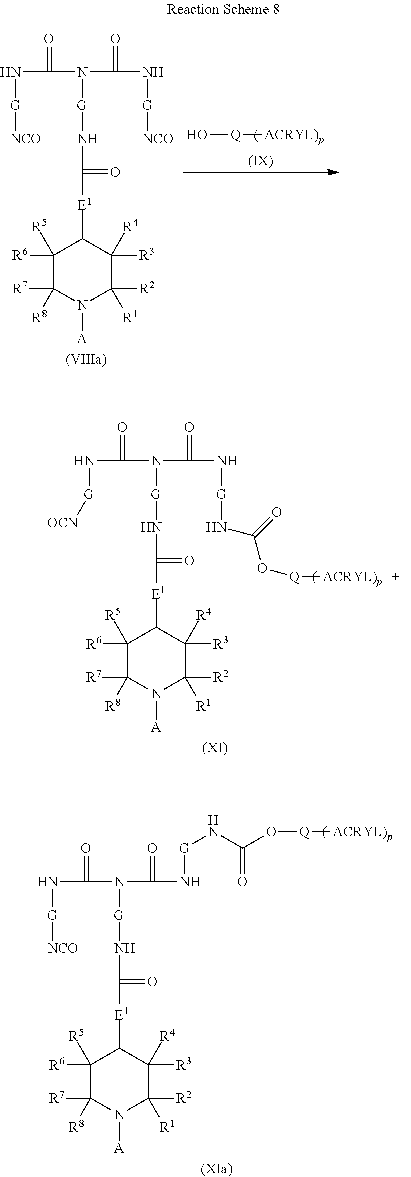

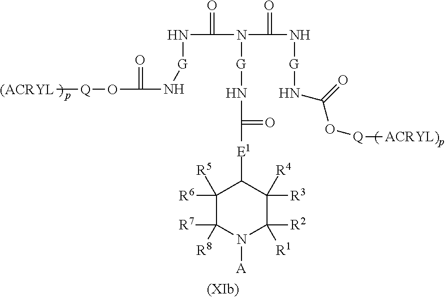

Compounds of Formulas (XI), (XIa), and (XIb) often feature R¹, R², R⁷, and R⁸ that are methyl, R³, R⁴, R⁵, R⁶ that are H. Such compounds are compounds of Formulas (XI1), (XIa1), and (XIb1).

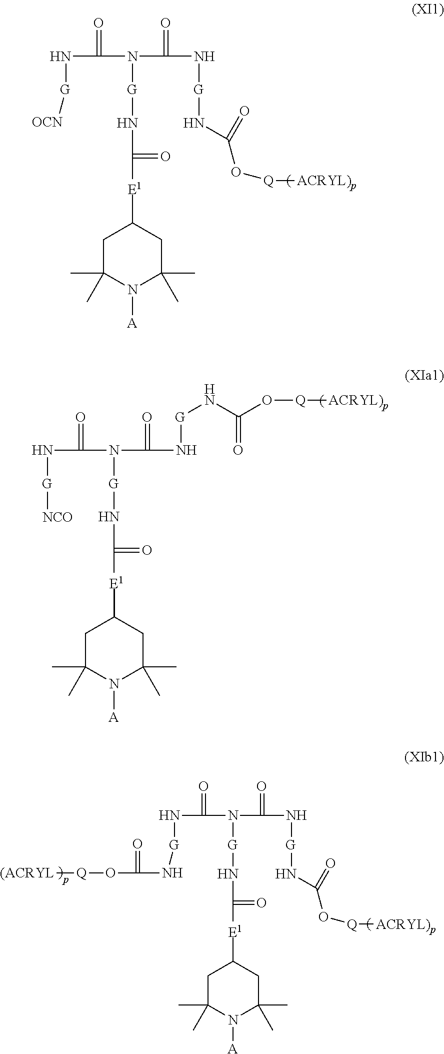

When E¹ in the compounds of Formulas (XI1), (XIa1), and (XIb1) is O, then those compounds are compounds of (XI2), (XIa2), and (XIb2), respectively.

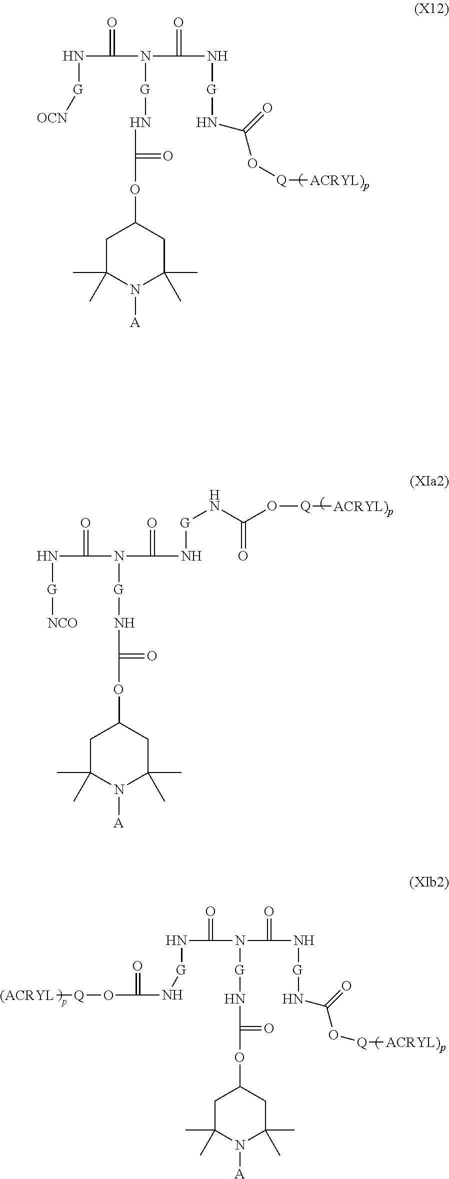

When E¹ in the compounds of Formulas (XI1), (XIa1), and (XIb1) is NH, then those compounds are compounds of (XI3), (XIa3), and (XIb3), respectively.

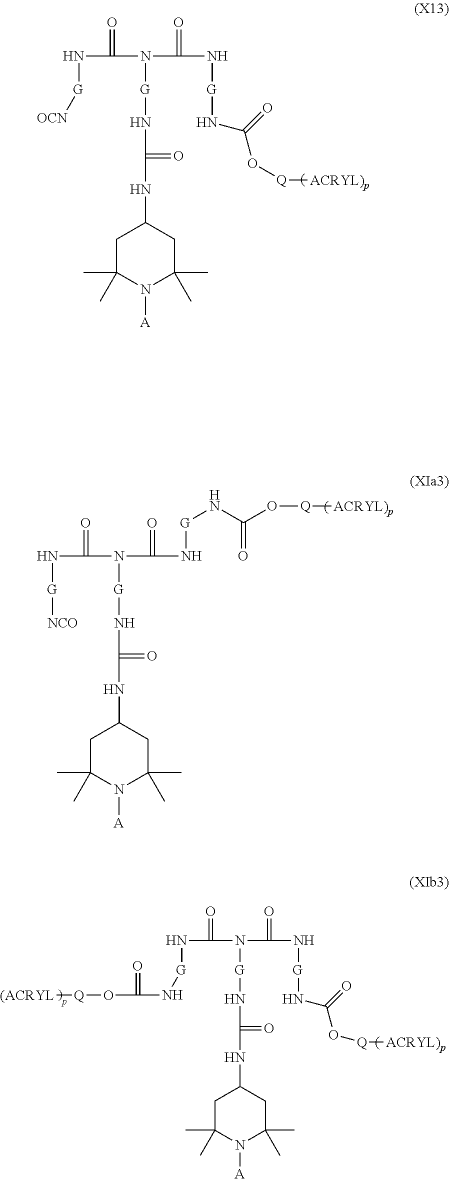

In any of the compounds of Formulas (XI2), (XIa2), (XIb3), (XI3), (XIa3), and (XIb3), E² is most often O. Likewise, Q is most commonly hydrocarbon polyradical, particularly C₁ to C₁₂ or C₁ to C₆ hydrocarbon polyradical. The hydrocarbon polyradical is often alkylene, in which case the alkylene is often C₁ to C₁₂ or C₁ to C₆ alkylene. G is alkylene, most often C₁ to C₁₂ alkylene, such as C₁ to C₆ alkylene. Hexylene is most common. ACRYL in these compounds typically features an R^{d} that is methyl or H.

A in any of the compounds of Formulas (XI2), (XIa2), (XIb3), (XI3), (XIa3), and (XIb3) can be alkyl or oxyalkyl. When alkyl is employed, the alkyl is typically C₁ to C₆ alkyl. Methyl is most common. When oxyalkyl is employed, the oxyalkyl is typically C₁ to C₁₂ oxyalkyl. Oxyoctyl is most common.

Reaction Scheme 9 shows the reactions of compounds of Formula (VIIb) and (VIIc) with the compound of Formula (IX) to give a compound of Formula (XII) or (XIIa), respectively. These reactions can be carried out using the same reaction conditions described above with respect to Reaction Scheme 7. Unlike the reactions shown in Reaction Schemes 7 and 8, each reaction of Reaction Scheme 9 gives only one product. The yield of the product can be highest when slightly more than one equivalent of the compound of Formula (IX) is used.

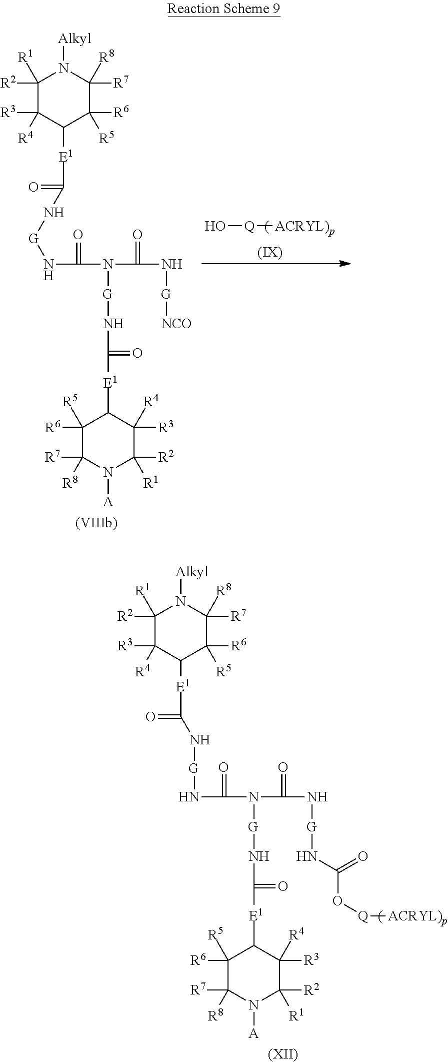

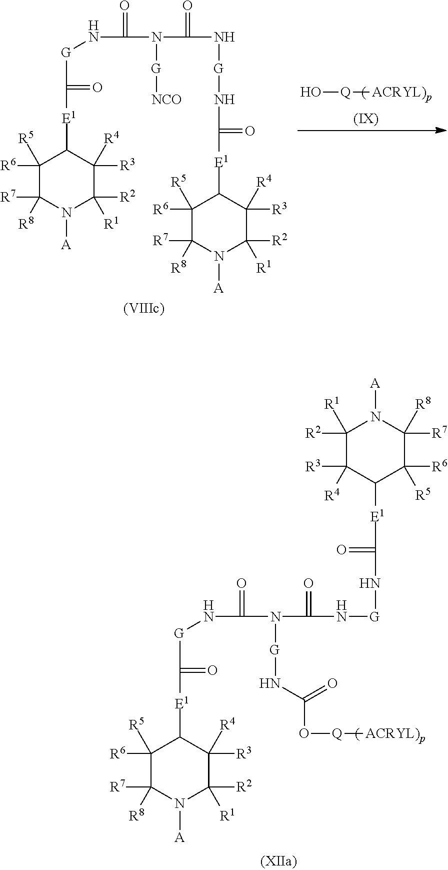

The compounds of Formulas (XII) and (XIIa) are compounds of Formula (I) wherein o is 2 and r is 1. In these compounds, the identity of E¹, A, R¹ through R⁸, and each G is carried over from the compound of Formula (VIII). Thus, the identity of any of these elements is the same as that described above with respect to Formula (VIII). Similarly, the identity of Q is carried over from the compound of Formula (IX), and is therefore the same as that described above with respect to the compound of Formula (IX).

Compounds of Formulas (XII) and (XIIa) often feature R¹, R², R⁷, and R⁸ that are methyl, R³, R⁴, R⁵, R⁶ that are H. Such compounds are compounds of Formulas (XIII) and (XIIa1).

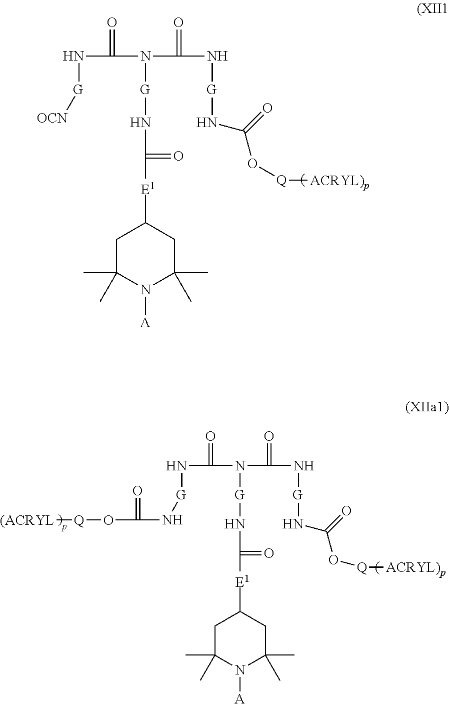

When E¹ in the compounds of Formulas (XII1) and (XIIa1) is O, then those compounds are compounds of Formula (XII2) and (XIIa2), respectively.

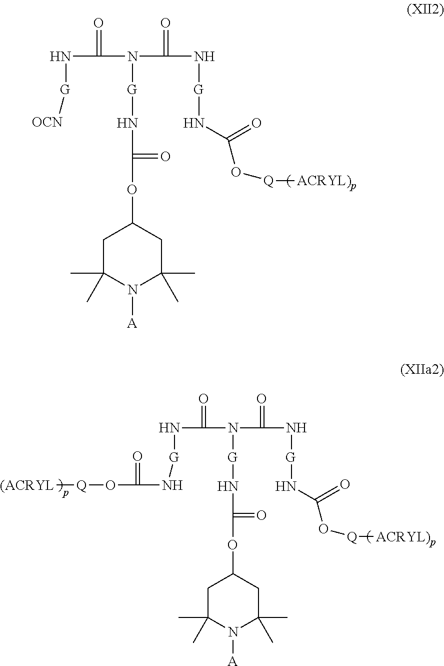

When E¹ in the compounds of Formulas (XIII) and (XIIa1) is NH, then those compounds are compounds of Formula (XII3) and (XIIa3), respectively.

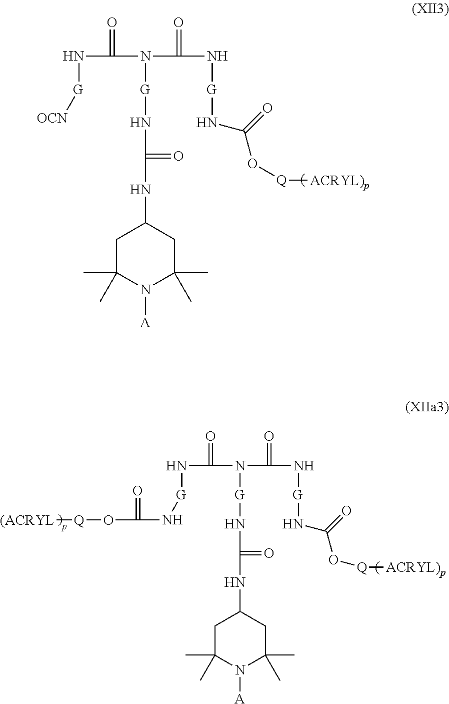

In compounds of Formulas (XII2), (XIIa2), (XII3), and (XIIa3), E² is most often O. Likewise, Q is most commonly hydrocarbon polyradical, particularly C₁ to C₁₂ or C₁ to C₆ hydrocarbon polyradical. The hydrocarbon polyradical is often alkylene, in which case the alkylene is often C₁ to C₁₂ or C₁ to C₆ alkylene. G is alkylene, most often C₁ to C₁₂ alkylene, such as C₁ to C₆ alkylene. Hexylene is most common.

A in any of the compounds of Formulas (XII2), (XIIa2), (XII3), and (XIIa3) can be alkyl or oxyalkyl. When alkyl is employed, the alkyl is typically C₁ to C₆ alkyl. Methyl is most common. When oxyalkyl is employed, the oxyalkyl is typically C₁ to C₁₂ oxyalkyl. Oxyoctyl is most common. ACRYL in these compounds typically features an R^{d} that is methyl or H

Other compounds of Formula (VII) can be used to form compounds of Formula (I). For example, a compound of Formula (VIIc), which is an exemplary di-isocyanate, can react with a compound of Formula (III) according to Reaction Scheme 10. In the compound of Formula (VIIc), G′ can be any suitable linking group. Typically, G′ is alkylene, such as C₁ to C₂₀ alkylene, but G′ can also have other structures such as

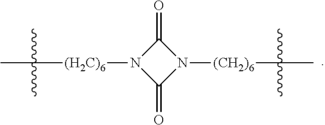

The reaction shown in Reaction Scheme 10 can take place under the conditions discussed above with respect to Reaction Scheme 6. The products of Reaction Scheme 10 are the compounds of Formulas (XIII) and (XIIIa). The compound of Formula (XIII) is a compound of Formula (I) wherein o is 2 and both r and 1 are 0. The compound of Formula (XIIIa) is a compound of Formula (I) wherein o is 1, r is 0 and q is 1. In these compounds, the identity of E¹, A, and R¹ through R⁸, is carried over from the compound of Formula (VIII). Thus, the identity of any of these elements is the same as that described above with respect to Formula (VIII). The identity of G′ is carried over from the compound of Formula (VIIc), and is identical as described above with respect to that Formula (VIIc).

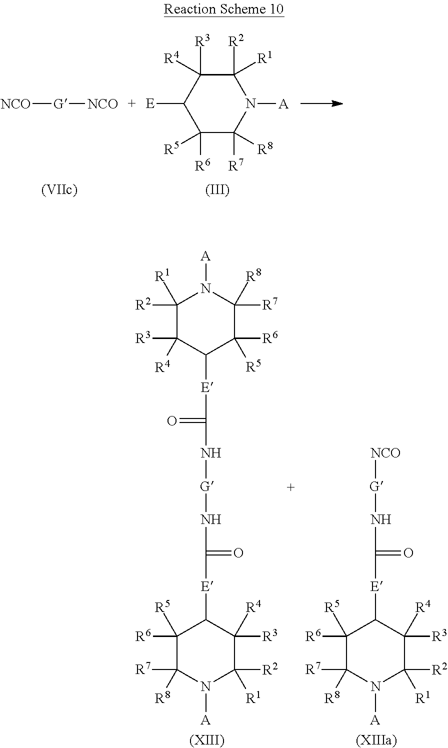

The compound of Formula (XIIIa) can further react with a compound of Formula (IX) according to Reaction Scheme 11. The products of Reaction Scheme 11 are compounds of Formulas (XIII) and (XIIIa). The compound of Formula (XIII) does not have any acrylate groups. The compound of Formula (XIIIa) can be a precursor to a compound of Formula (I).

The compound of Formula (XIIIa) can react with a compound of Formula (IX) to form a compound of Formula (XIV), which is a compound of Formula (I) wherein o and r are each 1 and q is 0. The reaction can be carried out under the same reaction conditions discussed above with respect to Reaction Scheme 9.

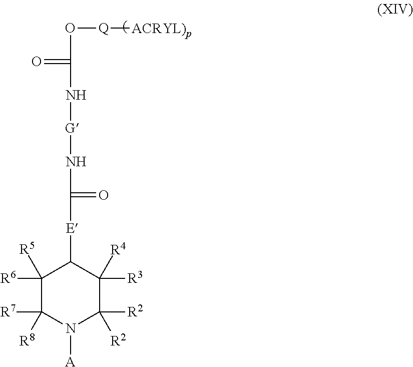

In the compound of Formula (XIV), the identity of E¹, A, R¹ through R⁸, and each G is carried over from the compound of Formula (VIII). Similarly, the identity of Q is carried over from the compound of Formula (IX), and is therefore the same as that described above with respect to the compound of Formula (IX).

A in any of the compounds of Formula (XIV), can be alkyl or oxyalkyl. When alkyl is employed, the alkyl is typically C₁ to C₆ alkyl. Methyl is most common. When oxyalkyl is employed, the oxyalkyl is typically C₁ to C₁₂ oxyalkyl. Oxyoctyl is most common. ACRYL in these compounds typically features an R^{d} that is methyl or H. R¹ through R⁸ in such compounds is defined as above with respect to Formula (I). Typically, R¹, R², R⁷, and R⁸ are methyl and R³ through R⁶ are H. E¹ is also defined as above with respect to Formula (I). G is defined as above with respect to Formula (VII). Hexylene is one common G, although others are also possible.

Compounds of Formula (I) can be prepared starting with any multi-isocyanate by using synthetic methodology that is analogous to that shown in Reaction Schemes 6, 7, 8, 9, and 10. Specifically, a compound of Formula (III), such as a compound of Formula (IIIa), (IIIb), (IV), or (IVa) can react with the multi-isocyanate to form hindered alkyl amine adduct or a hindered oxyalkyl amine adduct. Compounds of Formulas (IIIa1), (IIIa2), (IIIb1), (IIIb2), (IV1), (IV2), (IV3), (IVa1), (IVa2), or (IVa3) are commonly used, in which case then the identity of R¹ through R⁸ and A correspond to that of the particular compound used.

The resulting adduct can further react with a compound of Formula (IX) to attach one or more (alkyl)acrylate groups to one or more of the remaining isocyanate moieties. Because the reactive chemical moieties are the same regardless of the identity of the multi-isocyanate, a person of skill in the art can carry out the chemical reactions using the guidance provided herein with respect to Schemes 6, 7, 8, 9, and 10. The identity of Rᵢ in any resulting compound of Formula (I) will depend on the specific isocyanate used.

In addition to the multi-isocyanates discussed above, any multi-isocyanate can be used as a starting material to provide compounds of Formula (I). For instance, a variety of di-isocynates other than those shown above are commonly used. Examples specific multi-isocyanates that can be used include those discussed in U.S. Pat. No. 7,718,264 at column 8, lines 10-26, and compounds of Formulas (XV), (XVa), (XVb), and (XVc), all of which are commercially available. For example, the compound of Formulas (XV), (XVa), (XVb), and (XVc), are obtainable under the trade designation DESMODUR N3600 (XV), DESMODUR N3900(XVa), DESMODUR N3400(XVb), and DESMODUR W(XVc), respectively, all of which are available from Bayer Polymers LLC (Pittsburgh, USA).

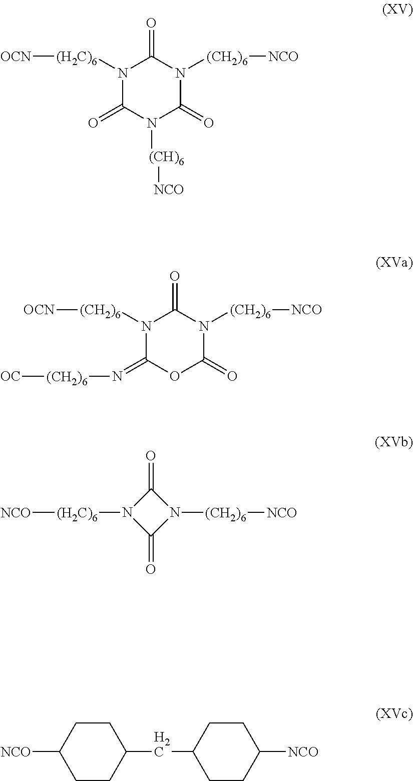

Any of the compounds of Formulas (XV), (XVa), (XVb), or (XVc) substituted for the compound of Formula (VIIa) in Reaction Scheme 6, and used to produce compounds of Formulas (I), (II), or (IIa) by following the procedures described above with respect to Reaction Schemes 6, 7, 8, and 9.

Any of the compounds described herein having a hindered amine light stabilizer component and at least one (alkyl)acrylate, isocyanate, or both, can be incorporated into the backbone of a polymer or copolymer, thereby providing a polymer or copolymer that is derived from the compound. For example, those compounds that contain an (alkyl)acrylate moiety can be incorporated into the backbone of an acrylic polymer or a polyolefin by copolymerizing the compound with an ethylenically unsaturated monomer, such as a acrylate or (meth)acrylate. Such copolymerization can take place by any process suitable for polymerizing the ethylenically unsaturated monomer. Exemplary methods include radical polymerization, anionic polymerization, and cationic polymerization.

Radical polymerization is most common. Radical polymerization is typically carried out by mixing the hindered amine light stabilizer compound containing an (alkyl)acrylate moiety with one or more ethylenically unsaturated monomers and one or more radical initiators. The radical initiator is then activated, allowing the formation of radicals and subsequent conversion of the hindered amine light stabilizer compound containing an (alkyl)acrylate moiety wand one or more ethylenically unsaturated monomers to polymer. The method of activating the radical initiator depends on the nature of the radical initiator employed. Some radical initiators, such as azobisisobutyronitrile, can be activated by heating, whereas other radical initiators, for example peroxides such as benzoyl peroxide and 2,2-dimethyoxy-2-phenylacetophenone, can be activated by exposure to actinic radiation. Typically, ultra violet radiation is used.

Exemplary radically polymerizable monomers and co-monomers that can be used as monomers or co-monomers for polymerization with the compounds discussed herein include methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]p-propoxyphenyldimethylmethane, ethoxylated bisphenolA di(meth)acrylate, and tri shydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.) can also be used.

Those compounds described herein that do not include an (alk)acrylate or isocyanate group can be used by blending with one or more polymers or copolymers. Standard techniques known in the art for blending polymers with polymer additives can be used. The compounds are often blended with a pre-polymer, which is then cured to form the final polymer or copolymer. The presence of the hindered amine light stabilizer compound as a polymer or copolymer additive mitigates the negative effects of actinic radiation, such as visible and UV light, on the polymer or copolymer.

When the polymer or copolymer is intended for use as a coating, the hindered amine light stabilizer compound containing an (alk)acrylate moiety, one or more ethylenically unsaturated monomers, and one or more initiators can first be coated onto a substrate. Subsequently, the mixture can be dried and cured to form high molecular weight polymer. In such cases, it is often convenient to use one or more photoinitiator that can be activated by light; use of one or more photoinitiators allows curing to be effected by exposing the coated substrate to actinic radiation, typically ultraviolet radiation.

Hindered amine light stabilizer compound containing an isocyanate moiety, and particularly those containing two or more isocyanate moieties, can be used as an isocyanate component to form polyurethanes. Methods for forming polyurethanes from such materials are known in the art and are described, for example, in U.S. Pat. No. 5,354,808 (Onwumere).

The compounds described herein that include neither an (alkyl)acrylate nor an isocyanate moiety can be blended with polymers, such as polyolefins, polyacrylics, polystyrene, polyurethanes, and the like, in order to mitigate the effects of actinic radiation, such as visible and ultraviolet light, on the polymer.

Articles, such as molded articles and coated articles, can comprise one or more of the polymers or copolymers described herein.

### List of Illustrative Embodiments

The following listing of embodiments illustrates particular features and aspects of the disclosure. The disclosure also encompasses embodiments not listed. This list is therefore not intended to be limiting. Instead, the scope of protection sought is limited only by the appended claims.

Embodiment 1 is a compound having the structure of Formula (I)

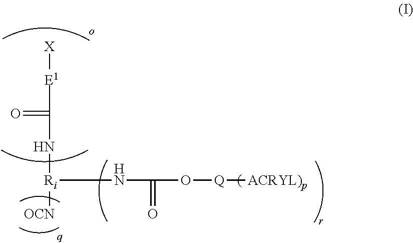

wherein

X is

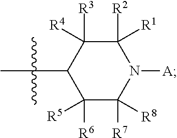

R¹ is alkyl,
R² is alkyl,
R³ is H or alkyl,
R⁴ is H or alkyl,
R⁵ is H or alkyl,
R⁶ is H or alkyl,
R⁷ is alkyl, and
R⁸ is alkyl;
A is alkyl or oxyalkyl;

Rᵢ is a residue of a multi-isocyanate;
E¹ is O or NR′,

R′ is H or C₁ to C₄ alkyl;
each Q is independently a connecting group having a valence of p+1;
each ACRYL is independently an (alk)acryl functional group of the formula OC(O)C(R^{d})═CH₂,

wherein

R^{d} is alkyl or H; and
p is the number of ACRYL groups attached to Q, which is from 1 to 6;
o is the number of

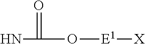

groups covalently bound to Rᵢ, which is from 1 to 8;
r is the number of

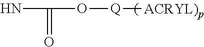

groups covalently bound to Rᵢ, which is from 1 to 8;
q is the number of NCO groups covalently bound to which is from 0 to 8;
and the sum of q+o+r is from 2 to 10.
Embodiment 2 is a compound of embodiment 1 wherein Rᵢ is

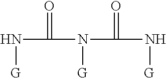

wherein
each G is independently alkylene that is also bound to

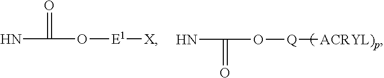

### or NCO; and

q+o+r is 3.
Embodiment 3 is a compound of embodiment 2 wherein G is C₁ to C₁₂ alkylene is 1.
Embodiment 4 is a compound of embodiment 2 wherein G is C6 alkylene.
Embodiment 5 is a compound of any of the preceding embodiments wherein ACRYL is OC(O)C(R^{d})═CH₂ and R^{d} is methyl or H.
Embodiment 6 is a compound of any of the preceding embodiments wherein R^{d} is methyl.
Embodiment 7 is a compound of any of the preceding embodiments wherein R^{d} is H.
Embodiment 8 is a compound of any of the preceding embodiments wherein Q is hydrocarbon polyradical.
Embodiment 9 is a compound embodiment 8 wherein the hydrocarbon polyradical is C₁ to C₁₂ hydrocarbon polyradical.
Embodiment 10 is a compound of embodiment 9 wherein the hydrocarbon polyradical is C₁ to C₆ hydrocarbon polyradical.
Embodiment 11 is a compound of any of embodiments 8-10 wherein the hydrocarbon polyradical is alkylene.
Embodiment 12 is a compound of any of the preceding embodiments wherein p is 1-3.
Embodiment 13 is a compound of embodiment 12 wherein p is 1.
Embodiment 14 is a compound of embodiment 12 wherein p is 2.
Embodiment 15 is a compound of embodiment 12 wherein p is 3.
Embodiment 16 is a compound of any of the preceding embodiments wherein E¹ is 0.
Embodiment 17 is a compound of any of embodiments 1-15 wherein E¹ is NH.
Embodiment 18 is a compound of any of the preceding embodiments wherein R¹ is C₁ to C₆ alkyl.
Embodiment 19 is a compound of embodiment 18 wherein R¹ is methyl.
Embodiment 20 is a compound of any of the preceding embodiments wherein R² is C₁ to C₆ alkyl.
Embodiment 21 is a compound of embodiment 20 wherein R² is methyl.
Embodiment 22 is a compound of any preceding embodiment wherein R³ is H.
Embodiment 23 is a compound of any preceding embodiment wherein R⁴ is H.
Embodiment 24 is a compound of any preceding embodiment wherein R⁵ is H.
Embodiment 25 is a compound of any preceding embodiment wherein R⁶ is H.
Embodiment 26 is a compound of any preceding embodiment wherein R⁷ is C₁ to C₆ alkyl.
Embodiment 27 is a compound of embodiment 26 wherein R⁷ is methyl.
Embodiment 28 is a compound of any preceding embodiment wherein R⁸ is C₁ to C₆ alkyl.
Embodiment 29 is a compound of embodiment 28 wherein R⁸ is methyl.
Embodiment 30 is a compound of any of embodiments 1 or 5-29 wherein R₁ is

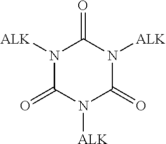

wherein each ALK is independently alkylene that is that is also bound to

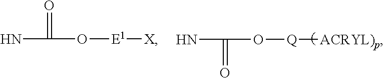

### or NCO.

Embodiment 31 is a compound of embodiment 30 wherein each ALK is C₁ to C₁₂ alkylene.
Embodiment 32 is a compound of embodiment 31 wherein each C₁ to C₁₂ alkylene is hexylene.
Embodiment 33 is a compound of any of embodiments 1-29 wherein R₁ is

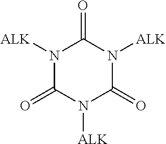

wherein each ALK is independently alkylene that is that is also bound to

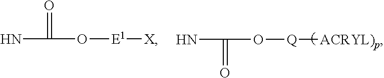

### or NCO.

Embodiment 34 is a compound of embodiment 33 wherein each alkylene is hexylene.
Embodiment 35 is a compound of any of embodiments 1-29 wherein Rᵢ is

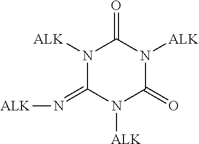

wherein each ALK is independently alkylene that is that is also bound to

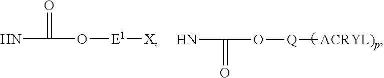

### or NCO.

Embodiment 36 is a compound of embodiment 35 wherein each alkylene is hexylene.
Embodiment 37 is a compound of any of embodiments 1-29 wherein Rᵢ is

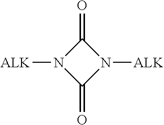

wherein each ALK is independently alkylene that is that is also bound to

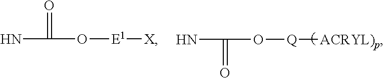

### or NCO.

Embodiment 38 is a compound of embodiment 37 wherein each alkylene is hexylene.
Embodiment 39 is a compound of any of embodiments 1-29 wherein Rᵢ is

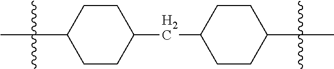 [SMILES: *C1CCC(CC2CCC(*)CC2)CC1]

wherein each ALK is independently alkylene that is that is also bound to

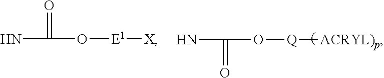

### or NCO.

Embodiment 40 is a compound of any of embodiments 1-3 or 4-29 wherein the compound is a compound of Formula (X).
Embodiment 41 is a compound of any of embodiments 1-3 or 4-29 wherein the compound is a compound of Formula (Xa).
Embodiment 42 is a compound of any of embodiments 1-3 or 4-29 wherein the compound is a compound of Formula (Xb).
Embodiment 43 is a compound of any of embodiments 1-3 or 4-29 wherein the compound is a compound of Formula (X1).
Embodiment 44 is a compound of any of embodiments 1-3 or 4-29 wherein the compound is a compound of Formula (Xa1).
Embodiment 45 is a compound of any of embodiments 1-3 or 4-29 wherein the compound is a compound of Formula (Xb 1).
Embodiment 46 is a compound of any of embodiments 1-3 or 4-29 wherein the compound is a compound of Formula (X2).
Embodiment 47 is a compound of any of embodiments 1-3 or 4-29 wherein the compound is a compound of Formula (Xa2).
Embodiment 48 is a compound of any of embodiments 1-3 or 4-29 wherein the compound is a compound of Formula (Xb2).
Embodiment 49 is a compound of any of embodiments 1-3 or 4-29 wherein the compound is a compound of Formula (X3).
Embodiment 50 is a compound of any of embodiments 1-3 or 4-29 wherein the compound is a compound of Formula (Xa3).
Embodiment 51 is a compound of any of embodiments 1-3 or 4-29 wherein the compound is a compound of Formula (Xb3).
Embodiment 52 is a compound of any of embodiments 1-3 or 4-29 wherein the compound is a compound of Formula (XI).
Embodiment 53 is a compound of any of embodiments 1-3 or 4-29 wherein the compound is a compound of Formula (XIa).
Embodiment 54 is a compound of any of embodiments 1-3 or 4-29 wherein the compound is a compound of Formula (XIb).
Embodiment 55 is a compound of any of embodiments 1-3 or 4-29 wherein the compound is a compound of Formula (XI1).
Embodiment 56 is a compound of any of embodiments 1-3 or 4-29 wherein the compound is a compound of Formula (XIa1).
Embodiment 57 is a compound of any of embodiments 1-3 or 4-29 wherein the compound is a compound of Formula (XIb 1).
Embodiment 58 is a compound of any of embodiments 1-3 or 4-29 wherein the compound is a compound of Formula (XI2).
Embodiment 59 is a compound of any of embodiments 1-3 or 4-29 wherein the compound is a compound of Formula (XIa2).
Embodiment 60 is a compound of any of embodiments 1-3 or 4-29 wherein the compound is a compound of Formula (XIb2).
Embodiment 61 is a compound of any of embodiments 1-3 or 4-29 wherein the compound is a compound of Formula (XI3).
Embodiment 62 is a compound of any of embodiments 1-3 or 4-29 wherein the compound is a compound of Formula (XIa3).
Embodiment 63 is a compound of any of embodiments 1-3 or 4-29 wherein the compound is a compound of Formula (XIb3).
Embodiment 64 is a compound of any of embodiments 1-3 or 4-29 wherein the compound is a compound of Formula (XII).
Embodiment 65 is a compound of any of embodiments 1-3 or 4-29 wherein the compound is a compound of Formula (XIIa).
Embodiment 66 is a compound of any of embodiments 1-3 or 4-29 wherein the compound is a compound of Formula (XIII).
Embodiment 67 is a compound of any of embodiments 1-3 or 4-29 wherein the compound is a compound of Formula (XIIa1).
Embodiment 68 is a compound of any of embodiments 1-3 or 4-29 wherein the compound is a compound of Formula (XII2)
Embodiment 69 is a compound of any of embodiments 1-3 or 4-29 wherein the compound is a compound of Formula (XIIa2).
Embodiment 70 is a compound of any of embodiments 1-3 or 4-29 wherein the compound is a compound of Formula (XII3).
Embodiment 71 is a compound of any of embodiments 1-3 or 4-29 wherein the compound is a compound of Formula (XIIa3).
Embodiment 72 is a compound of any of embodiments 1-3 or 4-29 wherein the compound is a compound of Formula (XIV).
Embodiment 73 is a compound of any of embodiments 1-3 or 4-29 wherein the compound is a compound of Formula (XIV1).
Embodiment 74 is a compound of any of embodiments 1-3 or 4-29 wherein the compound is a compound of Formula (XIV2).
Embodiment 75 is a compound of any of embodiments 1-3 or 4-29 wherein the compound is a compound of Formula (XIV3).
Embodiment 76 is a compound of any of embodiments 1-32 wherein o is 1-3.
Embodiment 77 is a compound of embodiment 76 wherein o is 1-2.
Embodiment 78 is a compound of embodiment 77 wherein o is 2
Embodiment 79 is a compound of embodiment 78 wherein o is 1.
Embodiment 80 is a compound of any preceding embodiment wherein q is 0-3.
Embodiment 81 is a compound of any preceding embodiment wherein q is 0-2.
Embodiment 82 is a compound of any preceding embodiment wherein q is 0-1.
Embodiment 83 is a compound of any preceding embodiment wherein q is 1.
Embodiment 84 is a compound of any of embodiments 1-39 wherein q is 0.
Embodiment 85 is a polymer or copolymer derived from a compound of any of the preceding embodiment.
Embodiment 86 is a coating comprising a compound of any of embodiments 1-84 or a polymer or copolymer or embodiment 85.
Embodiment 87 is a coated substrate, wherein the coating comprises the coating of embodiment 86.
Embodiment 88 is an article comprising a compound of any of embodiments 1-84, a polymer or copolymer of embodiment 85, a coating of embodiment 86, or a substrate of embodiment 87.
Embodiment 89 is an article of embodiment 88, wherein the article is a coated article.
Embodiment 90 is an article of any of embodiments 88-89, wherein the article is a molded article.

### EXAMPLES

Materials

1,2,2,6,6-pentamethyl-4-hydroxy-piperidine (PMHP) was obtained from TCI America (Portland, Oreg., USA.)

TINUVIN 123, IRGACURE 184, and IRGACURE 819 were obtained from BASF (Florham Park, USA) under trade designations “TINUVIN 123”, “IRGACURE 184”, and “IRGACURE 819” respectively.

1,1-bis(acryloyloxymethyl)ethyi isocyanate (BET), isocyanatoethyl acrylate (AOI), and isocyanatoethyl methacrylate (MOI, also designated as IEM), were obtained from obtained CBC America Corp (Commack, N.Y.)

TEGORAD 2100 was obtained from Evonik (Piscataway,USA) under trade designation “TEOGRAD 2100”.

Tetrahydrofuran (THF), methyl ethyl ketone (MEK), methyl t-butyl ether (MTBE), sodium carbonate, sodium hydroxide, anhydrous magnesium sulfate, 85% potassium hydroxide, dimethyl sulfoxide (DMSO), methylene chloride (dichloromethane), methanol, chloroform, and triethylamine were obtained from EMD Chemicals (Gibbstown, USA.)

Hydroxyethyl acrylate (HEA), 4-methoxyphenol (MEHQ), triethylamine, dibutyltin dilaurate (DBTDL), acryloyl chloride, oxalyl chloride, and sodium cyanoborohydride were obtained from Sigma-Aldrich (Milwaukee, USA.)

Ammonium acetate was obtained from VWR (West Chester, USA.)

EBECRYL 600 (epoxy acrylate of the diglycidyl ether of bisphenol A), was obtained from Allnex, (Alpharetta USA) under trade designation “EBECRYL 600”.

DESMODUR N100 and DESMODUR N 3600 were obtained from Bayer Polymers LLC, of Pittsburgh, Pa. under trade designation “DESMODUR N100” and “DESMODUR N 3600” respectively.

Pentaerythritol triacrylate (PET3A) was obtained from Sartomer Company of Exton, Pa. under the designation “SR444C”.

Hexanediol diacrylate was obtained from Sartomer Company of Exton, Pa. under the designation “SR238”.

Acrylated benzotriazole CAS number 96478-09-0, was obtained from TCI America, Portland, Oreg.

4-hydroxytempo was obtained from BASF in Florham Park, N.J. under trade designation “PROSTAB 5198”.

1-methoxy-2-propanol was obtained from Alfa Aesar in Ward Hill, Mass.

### Preparative Example 1

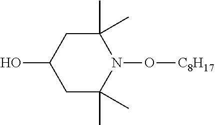

A 1 L 3-necked round bottom equipped with overhead stirrer and a vacuum bearing was charged with 200 g (0.275 mol, 0.55 eq, 737 MW) TINUVIN 123, and 323 g ethanol and placed in an oil bath at 70° C. To the reaction was added 73.23 g (1.109 mol, 66.01 MW) 85% potassium hydroxide. As the base was added the color of the reaction mixture changed from yellow to orange to brown; the reaction mixture also began refluxing. The bottom of the flask was scraped to provide a homogeneous mixture.

After the reaction mixture refluxed for 3.5 hours, the flask was fitted with a distillation head and condenser and placed under aspirator vacuum. 215 g of ethanol was collected by distillation, after which the reaction mixture was a thick, taffy-like mass. 250 g of water was added to the reaction mixture and the inside of the flask was scraped to disperse or dissolve the solids. The mixture was stirred for about 10 min of stirring at about 50° C., after which 300 g MTBE was added to the flask and stirred for a further 10 min. The reaction mixture was then poured into a 2 L separatory funnel, the bottom layer drained off and the top layer washed with 250 g water in the funnel. After removing the aqueous layer, the organic layer was dried over anhydrous magnesium sulfate, filtered, and concentrated on a rotary evaporator under aspirator pressure at 90° C. for 2 h to provide 137.2 g (87%) of undistilled product. This was distilled at 140° C. (pot temperature) at 29.3 Pa to provide 127.5 g (80.8%) of product.

### Preparative Example 2

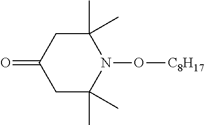

To a 500 mL 3-neck flask equipped with an overhead stirrer and nitrogen inlet adapter, and rubber septum was charged 12.04 g (0.1541 mol) dimethyl sulfoxide and 226 g of methylene chloride. The reaction was put under a nitrogen atmosphere and placed in an isopropanol dry-ice bath. After a few minutes, 9.78 g (0.0770 mol) oxalyl chloride was added via syringe through the septum over one minute. Five minutes later 20.00 g (0.0701 mol, approximate molecular weight 285.47) 2,2,6,6-tetramethyl-4-hydroxy-1-octyloxy-piperidine (the product of Preparative Example 1) was slowly added by syringe through the septum over 15 minutes. After a further 15 minutes of stirring, 17.72 g (0.17515 mol) triethylamine was added by syringe over about 30 seconds. Stirring was continued for 10 minutes in the isopropanol-dry ice bath, followed by a further 10 minutes at room temperature. The resulting solution was washed with 333 mL of 2-N hydrochloric acid, providing a mixture with distinct organic and aqueous layers. The organic and aqueous layers were separated, and the aqueous layer was extracted with 200 g of chloroform. The chloroform was combined with the other organic layer, and combined organic layers were dried over anhydrous magnesium sulfate, filtered, and concentrated on a rotary evaporator under water aspirator pressure at about 65° C. for 2 hours to provide an oil. The product was evaluated by ¹H NMR and FTIR, which gave results consistent with the expected structure.

### Preparative Example 3

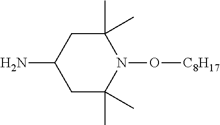

A 250 mL 3-necked flask equipped with overhead stirrer was charged with 5.00 g (0.017639 mol) 2,2,6,6-tetramethyl-4-keto-1-octyloxy-piperidine (the product of Preparative Example 2), 8 g of 3 angstrom molecular sieves, 13.60 g (0.17639 mol) ammonium acetate, and 77.5 g methanol and stirred for 1.75 hour under nitrogen at room temperature, after which 1.51 g (0.0242 mol) sodium cyanoborohydride in 13 g methanol was added to the reaction over 45 minutes and allowed to stir overnight. 360 g chloroform was then added to the reaction mixture and the mixture was washed twice with 400 g of 1N sodium hydroxide, dried over anhydrous magnesium sulfate, filtered, and concentrated at 40° C. at aspirator pressure on a rotary evaporator. Analysis by ¹H NMR showed the reaction to be a mixture of about 70 mole percent of the desired amine, 18 mole percent of a secondary amine, and 12 mole percent of the starting material. The products were separated to flash chromatography using an Analogix Intelliflash 280 from Agilent Technologies, Inc., Santa Clara, Calif. with a 150 g, 40 mm diameter column using a gradient of 25-30% methanol in methylene chloride over 20 minutes and then 30% methanol in methylene chloride to provide the desired product (2,2,6,6-tetramethyl-4-amino-1-octyloxy-piperidine) as an oil.

### Preparative Example 4

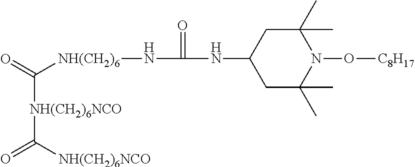

A 100 mL round bottom flask equipped with a magnetic stir bar was charged with 25.83 g (0.1345 eq, 192 EW) DESMODUR N100 and 30.00 g THF. The mixture was then swirled to dissolve the DESMODUR N100. A 25 mL pressure equalizing addition funnel was charged with 12.67 g (0.0444 eq, 285.47 EW) of the product of Preparative Example 3. The round bottom flask was placed in an ice bath and fitted with the addition funnel under dry nitrogen. The product of Preparative Example 3 was then added dropwise over 10 minutes with magnetic stirring, the addition funnel was then rinsed with 8.50 g THF. The reaction was monitored by FTIR and showed NCO absorption at 2265 cm⁻¹ as static after 19 hours. At 19.5 hours 0.019g DBTDL was charged to the reaction and the material was adjusted to 50 wt. % solids in THF.

### Example 1

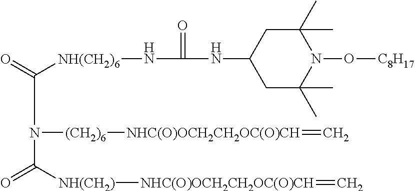

A 59 mL amber jar equipped with magnetic stir bar was charged with 10 g (0.0175 eq) of the product of Preparative Example 4, 8.5 microliters of a 10% solution by weight of DBTDL in THF, and 1.36 g (0.0117 eq, 116.12 EW) hydroxyethyl acrylate. The reaction solution was then magnetically stirred at 55° C. At 45 minutes 1.36 g tetrahydrofuran was charged to the jar. The reaction was monitored by FTIR and at 1 hour 45 minutes the material showed no NCO absorption at 2265 cm⁻¹. The material was then adjusted to 30% solids, 30% tetrahydrofuran and 40% isopropanol by charging 8.48 g to the jar.

### Example 2

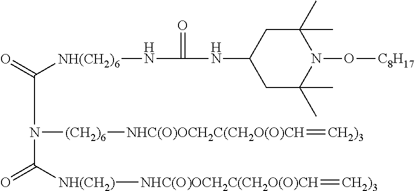

A 2 oz (59 mL) amber jar equipped with magnetic stir bar was charged with 20 g (0.0234 eq) DESMODUR N100/0.33NORHALS amine solution of Preparative Example 4, 72 microliters of a 10% solution by weight of DBTDL in THF, and 11.57 g (0.0234 eq, 494.3 EW) pentaerythritol triacrylate. The reaction solution was then magnetically stirred for 45 minutes in a 55° C. water bath, and then 11.57 g THF was charged to the jar. The reaction continued mixing and was monitored by FTIR; at 1 hour 45 minutes the material showed no NCO absorption at 2265 cm⁻¹. The material was then adjusted to 50% solids in THF, half was removed from the jar for later use and the remaining half was adjusted to 30% solids, 30% tetrahydrofuran and 40% isopropanol by charging 14.38 g isopropanol to the jar.

### Example 3

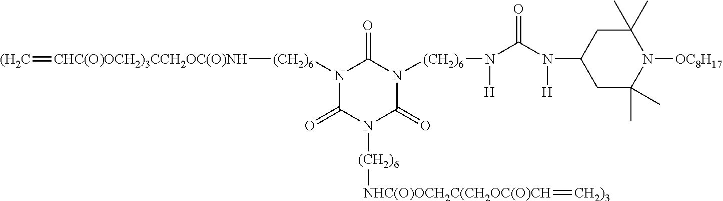

A 59 mL amber jar equipped with magnetic stir bar was charged with 2.87 g (0.0159 eq) DESMODUR N3600 followed by 2.87 g THF. Then 32 microliters of a 10% solution of DBDTL in THF was added, followed by 3.00 g (0.0052 eq, 286.47 EW) of the product of Preparative Example 3, added dropwise over one minute. The solution was magnetically stirred in a 55° C. water bath for ten minutes. Next 5.25 g (0.0106 eq, 494.3 EW) pentaerythritol triacrylate was charged to the jar, immediately followed by 5.25 g THF. The solution was then mixed overnight. The reaction was monitored by FTIR and the following morning the material showed no NCO absorption at 2265 cm⁻¹.

### Example 4

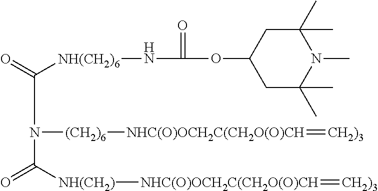

A 59 mL amber jar equipped with magnetic stir bar was charged with 2 g (0.0104 eq) DESMODUR N100 followed by 2 g THF. Then 1.18 g (0.0034 eq, 171.28 EW) of a 50% solution of the compound of Formula (IIIa2) in THF and 22 microliters of a 10% by weight solution of DBTDL in THF was added. The solution was magnetically stirred for 4.5 hours in a 55° C. water bath. Then 3.45 g (0.0070 eq, 494.3 EW) pentaerythritol triacrylate was charged to the jar, immediately followed by 3.45 g THF. The solution then continued mixing overnight. The reaction was monitored by FTIR and the following morning the material showed no NCO absorption at 2265 cm⁻¹. The material was then adjusted to 30% solids, 30% tetrahydrofuran and 40% isopropanol by charging 8.45 g isopropanol to the jar.

### Example 5

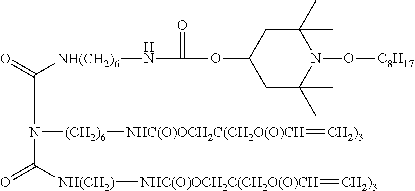

A 59 mL amber jar equipped with magnetic stir bar was charged with 2 g (0.0104 eq) DESMODUR N100 followed by 2 g THF. Then 1.96 g (0.0034eq, 284.47 EW) of a 50% solution of the compound of Formula (IIIb2) in THF and 22 microliters of a 10% by weight solution of DBTDL in THF was added. The solution was magnetically stirred for 4.5 hours in a 55° C. water bath. 3.45 g (0.0070 eq, 494.3 EW) pentaerythritol triacrylate was charged to the jar, immediately followed by 3.45 g THF. The solution continued mixing overnight at which point the reaction was shown by FTIR to be complete due to absence of NCO absorption at 2265 cm⁻¹. The material was then adjusted to 30% solids, 30% tetrahydrofuran and 40% isopropanol by charging 9.00 g isopropanol to the jar.

### Example 6

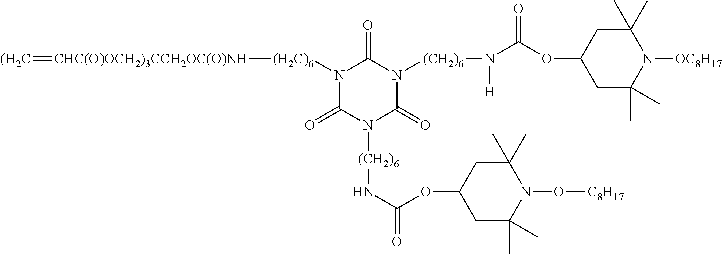

A 59 mL amber jar equipped with magnetic stir bar was charged with 6 g (0.0166 eq) of a 50% solution of DESMODUR N3600 in THF. Then 6.36 g (0.0111 eq, 286.47 EW) of a 50% solution of Formula (IIIb2) in THF was added, as well as 56 microliters of a 10% solution of DBTDL in THF. The solution was magnetically stirred overnight in a 55° C. water bath. 2.70 g (0.0055 eq, 494.3 EW) pentaerythritol triacrylate was charged to the jar, immediately followed by 2.70 g THF. The reaction was monitored by FTIR, after 4.5 hours of mixing the material showed no NCO absorption at 2265 cm⁻¹. The material was then adjusted to 30% solids, 30% tetrahydrofuran and 40% isopropanol by 12.43 g isopropanol to the jar.

1. A compound having the structure of Formula (I): 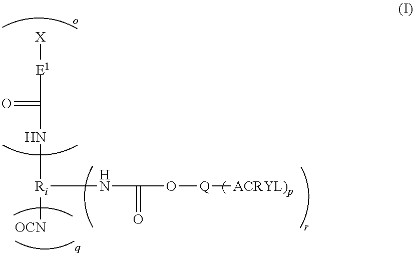
wherein
X is 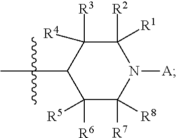
R¹ is alkyl,
R² is alkyl,
R³ is H or alkyl,
R⁴ is H or alkyl,
R⁵ is H or alkyl,
R⁶ is H or alkyl,
R⁷ is alkyl, and
R⁸ is alkyl;
A is alkyl or oxyalkyl;
Rᵢ is a residue of a multi-isocyanate;
E¹ is O or NR′,
R′ is H or C₁ to C₄ alkyl;
each Q is independently a connecting group having a valence of p+1;
each ACRYL is independently an (alk)acryl functional group of the formula OC(O)C(R^{d})═CH₂,
wherein
R^{d} is alkyl or H; and
p is the number of ACRYL groups attached to Q, which is from 1 to 6;
o is the number of 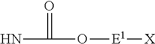
groups covalently bound to Rᵢ, which is from 1 to 8;
r is the number of 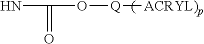
groups covalently bound to Rᵢ, which is from 1 to 8;
q is the number of NCO groups covalently bound to Rᵢ, which is from 0 to 8;
and the sum of q+o+r is from 2 to 10.

2. The compound of claim 1, wherein Rᵢ is 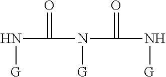
wherein
each G is independently alkylene that is also bound to 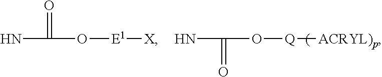
or NCO.

3. The compound of claim 2, wherein each G is C₁ to C₆ alkylene.

4. The compound of claim 2, wherein each G is C₁ to C₆ alkylene.

5. The compound of claim 1, wherein Rᵢ is selected from the group consisting of 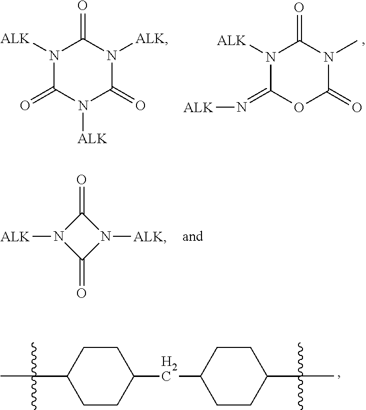
wherein each ALK is independently alkylene that is also bound to 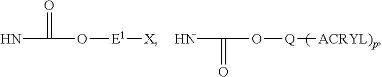
or NCO.

6. The compound of claim 1, wherein o is 1-3.

7. The compound of claim 1, wherein each q is hydrocarbon polyradical.

8. The compound of claim 1, wherein q+o+r is from 2 to 5.

9. The compound of claim 1, wherein q+o+r is from 2 to 3.

10. The compound of claim 1, wherein each E¹ is NH.

11. The compound of claim 1, wherein each E¹ is O.

12. The compound of claim 1, wherein p is 1-2.

13. The compound of claim 1, wherein r is 1-2.

14. The compound of claim 1, wherein q is 0.

15. A polymer or copolymer derived from or blended with a compound of claim 1.

16. An article comprising a compound of claim 1 or a polymer of claim 15.
